(19) United States
(12) Patent Application Publication
(10) Pub. No.: US 2024/0331837 A1
(43) Pub. Date: Oct. 3, 2024

(54) SELECTION OF VIRAL-SPECIFIC CYTOTOXIC T-CELL LINES FOR THE MANAGEMENT OF VIRAL INFECTIONS

(71) Applicant: BOARD OF REGENTS, THE UNIVERSITY OF TEXAS SYSTEM, Austin, TX (US)

(72) Inventors: Katy REZVANI, Houston, TX (US); David MARIN COSTA, Houston, TX (US); Elizabeth J. SHPALL, Houston, TX (US)

(73) Assignee: BOARD OF REGENTS, THE UNIVERSITY OF TEXAS SYSTEM, Austin, TX (US)

(21) Appl. No.: 18/579,223

(22) PCT Filed: Jul. 20, 2022
(86) PCT No.: PCT/US2022/073913

§ 371 Date: Jan. 12, 2024

Related U.S. Application Data
(60) Provisional application No. 63/224,694, filed on Jul. 22, 2021.

(51) Int. Cl. G16H 20/40; G16B 20/20

(52) U.S. Cl. CPC G16H 20/40; G16B 20/20

(57) ABSTRACT

Embodiments of the disclosure concern systems, methods, and compositions related to identification of a suitable cell therapy for an individual in need thereof. A cell line is selected from a plurality of cell lines for suitability for an individual based on an algorithm and cell identification number that includes values for HLA matching and the frequency of reactive CD4 and CD8 cytotoxic T lymphocytes as measured by interferon-γ and IL-2. Particular parameters for HLA matching and reactive frequencies are utilized to calculate points towards the cell identification number, and the cell line with the highest cell identification number may be utilized.

This application claims priority to U.S. Provisional Patent Application Ser. No. 63/224,694, filed Jul. 22, 2021, which is incorporated by reference herein in its entirety.

### BACKGROUND

### I. Technical Field

Embodiments of the disclosure relate at least to the fields of immunology, cell biology, molecular biology, and medicine.

### II. Background

Viral-specific cytotoxic T-cells (CTLs) are highly effective therapy for the management of viral infections, such as adenovirus, cytomegalovirus, JC virus, Epstein-Bar virus, Herpes virus 6, BK virus and SARS-CoV2. Viral-specific CTLs are particularly useful in the management of viral infections in severely immunocompromised patients as their administration results in the restoration of anti-viral immunity. Viral-specific CTLs are manufactured, cryopreserved, and stored in biobanks that normally contain multiple viral-specific CTL lines against each viral infection.

The present disclosure satisfies a long-felt need in the art of selecting CTL lines that are best suited for an individual in need thereof.

### BRIEF SUMMARY

Embodiments of the disclosure concern methods, compositions, and systems related to cell therapy for an individual and selection of an appropriate allogeneic cell line for the therapy. The methods, compositions, and systems increase the likelihood of the cells being therapeutically effective for a recipient individual and avoid deleterious immune responses upon administration of the cells to the individual. Specifically, the disclosure facilitates selection of a particular T cell line (including cytotoxic T lymphocytes, CTLs) for therapy for an individual. Each of the cell lines, or a plurality of cell lines, may recognize at least one epitope of an antigen (including a cancer antigen) or a virus. The individual in particular may be in need of viral-specific T cell therapy or cancer-specific T cell therapy, including therapy for the virus for which the T cells are directed or the cancer for which the T cells are directed against an associated antigen.

In at least some cases, the present disclosure provides a set of mathematical formulas that identify the best available T cell line for each individual in need thereof. Briefly, a database contains information regarding a plurality of available CTL lines in a given biobank or group of biobanks. The tissue type (HLA) of an individual in need of the T cells, and optionally one or more other variables, are utilized in a computer program that identifies the best available T cell line for therapy for the individual. The methods facilitate selection of suitable T cells from a plurality of T cells from which to choose. In various embodiments, the methods facilitate selection of a suitable T cell by identifying one or a plurality of HLA alleles (including combinations thereof) and also by identifying certain one or more relative activities of the T cell lines.

In certain embodiments, the methods of selecting an allogeneic T cell line further comprise prior to the selecting step, a step of HLA typing of the T cells and/or HLA typing of the cells in an intended recipient individual (such as typing any of the individual's cells, including blood cells, (e.g., including a mixture of B-cells, T-cells, neutrophils, and/or monocytes) or cells from a buccal swab). In certain embodiments, the methods of selecting an allogeneic T cell line further comprise, prior to a selecting step, a step of measuring the relative activities of a plurality of the cell lines, including measuring levels of one or more particular secreted proteins from the cells, such as secreted proteins that are associated with reactive frequencies of the cells, for example. A step of HLA typing of a cell line may or may not be performed prior to manufacturing of the cell line. A step of typing HLA alleles for a given T cell line may or may not occur before a step of determining reactive frequencies for the same cell line.

Embodiments of the disclosure include methods, comprising: receiving, by a processor, patient information representative of one or more human leukocyte antigen (HLA) genes for a patient; and determining, by the processor using an algorithm, a viral-specific cytotoxic T-cell line suitable for administering to the patient based on a cell line identification number for each of a plurality of cell lines that may be selected for the patient, and based on information about the HLA gene(s) indicated in the patient information. In some cases, the determining step is further defined as determining a viral-specific cytotoxic T-cell line suitable for administering to the patient based on values representative of matching of HLA gene(s) of the patient with respect to corresponding HLA gene(s) in the plurality of cell lines. The information about the HLA gene(s) for the patient may comprise, for each of the two chromosomes, the identity of the HLA gene, the HLA group, and the specific HLA protein. In specific embodiments, the information of the HLA gene(s) for the patient is represented by the following expression: HLA-X*YY:ZZ, where X is an HLA gene, YY is an HLA group, and ZZ is a specific HLA protein within the group. The information of the HLA gene(s) may be determined by a binary selection at YY and ZZ, said binary selection representing an allele from each of two chromosomes of the patient. In some cases in the formula, X represents HLA-A, HLA-B, HLA-C, or HLA-DR. The patient information about the HLA gene may comprise information with respect to a binary selection at YY and ZZ for each of HLA-A, HLA-B, HLA-C, and HLA-DR. In specific embodiments, the determining by the processor of a viral-specific cytotoxic T-cell line suitable for administering to the patient based on the cell line identification number is further defined as identifying a T-cell line having the same HLA allele for one or both chromosomes for one or more of HLA-A, HLA-B, HLA-C, and HLA-DR. In certain embodiments, determining by the processor the viral-specific cytotoxic T-cell line from a plurality of cell lines that may be selected for the patient comprises receiving information about a CD4+ or CD8+ dominant response based on information about an HLA gene for the patient. In particular embodiments, when the virus corresponding to the viral-specific cytotoxic T cell line is CD4 dominant, matching at HLA-DR has a weighted value for the algorithm that is greater than a weighted value for matching at HLA-B and/or a weighted value for matching at HLA-C. In other embodiments, when the virus corresponding to the viral-specific cytotoxic T cell line is CD4 dominant, matching at HLA-A has a weighted value for the algorithm that is greater than a weighted value for matching at HLA-B and/or a weighted value for matching at HLA-C. In some embodiments, when the virus corresponding to the viral-specific cytotoxic T cell line is CD4 dominant, the combination of matching at one allele for one chromosome at HLA-DR and matching at one allele for one chromosome at HLA-A has a weighted value that is greater than a weighted value for the combination of matching at each allele for both chromosomes of HLA-A or for both chromosomes at HLA-DR. When the virus corresponding to the viral-specific cytotoxic T cell line is CD4 dominant, matching at HLA-DR in the absence of concomitant matching at HLA-A may have a weighted value that is greater than a weighted value for matching at HLA-A with no concomitant matching at HLA-DR. In some cases, when the virus corresponding to the viral-specific cytotoxic T cell line is CD8 dominant, matching at HLA-DR has a weighted value for the algorithm that is greater than a weighted value for matching at HLA-B and/or a weighted value for matching at HLA-C. When the virus corresponding to the viral-specific cytotoxic T cell line is CD8 dominant, matching at HLA-A may have a weighted value for the algorithm that is greater than a weighted value for matching at HLA-B and/or a weighted value for matching at HLA-C.

In specific embodiments, when the virus corresponding to the viral-specific cytotoxic T cell line is CD8 dominant: the combination of (a) matching at one allele for HLA-DR and (b) matching at one allele for HLA-A has a weighted value in the algorithm that is greater than a weighted value for (1) or (2): (1) the combination of (a) matching at both alleles of HLA-A and (b) matching at no alleles at HLA-DR, or (2) the combination of (a) matching at both alleles at HLA-DR and (b) matching at no alleles at HLA-A. In some cases, when the virus corresponding to the viral-specific cytotoxic T cell line is CD8 dominant, matching at HLA-DR in the absence of concomitant matching at HLA-A has a weighted value that is less than a weighted value for matching at HLA-A with no concomitant matching at HLA-DR.

The method may further comprise: receiving, by the processor, information representative of a frequency of reactive cytotoxic T-lymphocyte (CTLs) from a plurality of cell lines that may be selected for the patient, wherein the CTLs comprise CD4+ cells and CD8+ cells and are measured by at least one of interferon-γ (IFN) secreted from CD4+ cells, IFN secreted from CD8+ cells, interleukin (IL)-2 secreted from CD4+ cells, and IL-2 secreted from CD8+ cells, wherein the step of determining, by the processor, the viral-specific cytotoxic T-cell line from a plurality of cell lines that may be selected for the patient is based on the frequency of reactive CTLs. In specific embodiments, the method further comprises subjecting a plurality of cells comprising CD4+ cells and CD8+ cells, from a plurality of cell lines that may be selected for the patient, to peptides from a viral antigen and identifying a proportion of the cells that are activated by the peptides. In certain aspects, the determining step is further defined as determining a proportion of CD4+ cells that are activated by the peptides and determining a proportion of CD8+ cells that are activated by the peptides. The activation may be determined by measuring the level of secretion of IL-2 and interferon-γ (IFN) from both of the CD4+ and CD8+ cell types. In some cases, the step of determining by the processor the viral-specific cytotoxic T-cell line from a plurality of cell lines that may be selected for the patient comprises identifying among CD4+ cells and CD8+ cells the proportion of CD4+ cells that secrete IL-2 and IFN and the proportion of CD8+ cells that secrete IL-2 and IFN. In various embodiments, when the virus corresponding to the viral-specific cytotoxic T cell line is CD4 dominant, then there may be a greater weighted value for the algorithm for the higher secretion level of CD4+ IFN or CD4+ IL-2. In various embodiments, when the virus corresponding to the viral-specific cytotoxic T cell line is CD8 dominant, then there may be a greater weighted value for the algorithm for the higher secretion level of CD8+ IFN or CD8+ IL-2. In specific embodiments, determining by the processor a viral-specific cytotoxic T-cell line suitable for administering to the patient based on the cell line identification number is based at least in part on one or more of the following values for when the response to the virus is CD4-dominant: (a) the product of the number of matches at HLA-DR times seven; (b) the product of the number of matches at HLA-A times six; (c) the number of matches at HLA-B; (d) the number of matches at HLA-C; (e) the number fifteen if there is a match at HLA-DR and HLA-A; (f) the number one if there is a match at HLA-B and HLA-C; (g) subtraction of one per integer when the frequency of reactive CD4+ or CD8+ cells is less than 10%; (h) subtraction of five when the frequency of reactive CD4+ or CD8+ cells is less than 3%; (i) subtraction of all points if the frequencies of reactive CD4+ or CD8+ cells are <1%. In some cases, based on the HLA match between the patient and the viral-specific cytotoxic T cell line, (g) and (h) are further defined as subtraction of one per integer: (a) based on the higher frequency of CD4+ cells that secrete IFN or IL-2; or (b) based on the higher frequency of CD8+ cells that secrete IFN or IL-2. In specific embodiments, determining by the processor a viral-specific cytotoxic T-cell line suitable for administering to the patient based on the cell line identification number is based at least in part on one or more of the following values for when the response to the virus is CD8-dominant: (a) product of the number of matches at HLA-A times seven; (b) product of the number of matches at HLA-DR times 6; (c) the number of matches at HLA-B; (d) the number of matches at HLA-C; (e) the number fifteen if there is a match at HLA-DR and HLA-A; (f) the number one if there is a match at HLA-B and HLA-C; (g) subtraction of one per integer when the frequency of reactive CD4+ or CD8+ cells is less than 10%; (h) subtraction of five when the frequency of reactive CD4+ or CD8+ cells is less than 3%; (i) subtraction of all points if the frequencies of reactive CD4+ or CD8+ cells are <1%. In some cases, based on the HLA match between the patient and the viral-specific cytotoxic T cell line, (g) and (h) are further defined as subtraction of one per integer: (a) based on the higher frequency of CD4+ cells that secrete IFN or IL-2; or (b) based on the higher frequency of CD8+ cells that secrete IFN or IL-2. In certain embodiments, for a virus in which it is unknown if they are CD4-dominant or CD8-dominant, one may perform studies in order to established what type of immune response those viruses elicit. In specific embodiments, for a virus in which it is unknown if they are CD4-dominant or CD8-dominant, one may select donors based on the CD8 T cell selection criteria over that for the CD4 T cell selection criteria.

In particular embodiments, a therapeutically effective amount of cells from the cell line having the highest cell line identification number based on the patient information is delivered to the patient.

Particular embodiments include an apparatus, comprising: a memory; and a processor coupled to the memory, in which the processor is configured to perform the steps comprising: receiving, by the processor, patient information representative of one or more human leukocyte antigen (HLA) genes for a patient; and determining, by the processor using an algorithm, a viral-specific cytotoxic T-cell line suitable for administering to the patient based on a cell line identification number for each of a plurality of cell lines that may be selected for the patient, and based on information about the HLA gene(s) indicated in the patient information. In specific embodiments, the determining step is further defined as determining a viral-specific cytotoxic T-cell line suitable for administering to the patient based on values representative of matching of HLA gene(s) of the patient with respect to corresponding HLA gene(s) in the plurality of cell lines. The information about the HLA gene(s) for the patient may comprise, for each of the two chromosomes, the identity of the HLA gene, the HLA group, and the specific HLA protein. The information of the HLA gene(s) for the patient may be represented by the following expression: HLA-X*YY:ZZ, where X is an HLA gene, YY is an HLA group, and ZZ is a specific HLA protein. In some cases, the information of the HLA gene(s) is determined by a binary selection at YY and ZZ, said binary selection representing an allele from each of two chromosomes of the patient. In some cases, in the formula, X represents HLA-A, HLA-B, HLA-C, or HLA-DR. The patient information about the HLA gene may comprise information with respect to a binary selection at YY and ZZ for each of HLA-A, HLA-B, HLA-C, and HLA-DR. In specific embodiments, the determining by the processor of a viral-specific cytotoxic T-cell line suitable for administering to the patient based on the cell line identification number is further defined as identifying a T-cell line having the same HLA allele for one or both chromosomes for one or more of HLA-A, HLA-B, HLA-C, and HLA-DR. In specific embodiments, determining by the processor the viral-specific cytotoxic T-cell line from a plurality of cell lines that may be selected for the patient comprises receiving information about a CD4+ or CD8+ dominant response based on information about an HLA gene for the patient. In some cases, when the response to the virus corresponding to the viral-specific cytotoxic T cell line is CD4 dominant, matching at HLA-DR has a weighted value for the algorithm that is greater than a weighted value for matching at HLA-B and/or a weighted value for matching at HLA-C. In some cases, when the response to the virus corresponding to the viral-specific cytotoxic T cell line is CD4 dominant, matching at HLA-A has a weighted value for the algorithm that is greater than a weighted value for matching at HLA-B and/or a weighted value for matching at HLA-C. In some cases, when the response to the virus corresponding to the viral-specific cytotoxic T cell line is CD4 dominant, the combination of matching at one allele for one chromosome at HLA-DR and matching at one allele for one chromosome at HLA-A has a weighted value that is greater than a weighted value for the combination of matching at each allele for both chromosomes of HLA-A or for both chromosomes at HLA-DR. In certain aspects, when the response to the virus corresponding to the viral-specific cytotoxic T cell line is CD4 dominant, matching at HLA-DR in the absence of concomitant matching at HLA-A has a weighted value that is greater than a weighted value for matching at HLA-A with no concomitant matching at HLA-DR. In some aspects, when the response to the virus corresponding to the viral-specific cytotoxic T cell line is CD8 dominant, matching at HLA-DR has a weighted value for the algorithm that is greater than a weighted value for matching at HLA-B and/or a weighted value for matching at HLA-C. In particular aspects, when the response to the virus corresponding to the viral-specific cytotoxic T cell line is CD8 dominant, matching at HLA-A has a weighted value for the algorithm that is greater than a weighted value for matching at HLA-B and/or a weighted value for matching at HLA-C. In specific aspects, when the response to the virus corresponding to the viral-specific cytotoxic T cell line is CD8 dominant: the combination of (a) matching at one allele for HLA-DR and (b) matching at one allele for HLA-A has a weighted value in the algorithm that is greater than a weighted value for (1) or (2): (1) the combination of (a) matching at both alleles of HLA-A and (b) matching at no alleles at HLA-DR, or (2) the combination of (a) matching at both alleles at HLA-DR and (b) matching at no alleles at HLA-A. In some embodiments, when the response to the virus corresponding to the viral-specific cytotoxic T cell line is CD8 dominant, matching at HLA-DR in the absence of concomitant matching at HLA-A has a weighted value that is less than a weighted value for matching at HLA-A with no concomitant matching at HLA-DR. The processor may be further configured to perform the steps comprising: receiving, by the processor, information representative of a frequency of reactive cytotoxic T-lymphocyte (CTLs) from a plurality of cell lines that may be selected for the patient, wherein the CTLs comprise CD4+ cells and CD8+ cells and are measured by at least one of interferon-γ (IFN) secreted from CD4+ cells, IFN secreted from CD8+ cells, interleukin (IL)-2 secreted from CD4+ cells, and IL-2 secreted from CD8+ cells, wherein the step of determining, by the processor, the viral-specific cytotoxic T-cell line from a plurality of cell lines that may be selected for the patient is based on the frequency of reactive CTLs. In some embodiments, the processor is further configured to perform the step comprising subjecting a plurality of cells comprising CD4+ cells and CD8+ cells, from a plurality of cell lines that may be selected for the patient, to peptides from a viral antigen and identifying a proportion of the cells that are activated by the peptides. The determining step may be further defined as determining a proportion of CD4+ cells that are activated by the peptides and determining a proportion of CD8+ cells that are activated by the peptides. In some cases, the activation is determined by measuring the level of secretion of interleukin (IL)-2 and IFN from both of the CD4+ and CD8+ cell types. The step of determining by the processor the viral-specific cytotoxic T-cell line from a plurality of cell lines that may be selected for the patient may comprise identifying among CD4+ cells and CD8+ cells the proportion of CD4+ cells that secrete IL-2 and IFN and the proportion of CD8+ cells that secrete IL-2 and IFN. In some cases, when the virus corresponding to the viral-specific cytotoxic T cell line is CD4 dominant, then there is a greater weighted value for the algorithm for the higher secretion level of CD4+ IFN or CD4+ IL-2. In particular embodiments, determining by the processor a viral-specific cytotoxic T-cell line suitable for administering to the patient based on the cell line identification number is based at least in part on one or more of the following values for when the response to the virus is CD4-dominant: (a) the product of the number of matches at HLA-DR times seven; (b) the product of the number of matches at HLA-A times six; (c) the number of matches at HLA-B; (d) the number of matches at HLA-C; (e) the number fifteen if there is a match at HLA-DR and HLA-A; (f) the number one if there is a match at HLA-B and HLA-C; (g) subtraction of one per integer when the frequency of reactive CD4+ or CD8+ cells is less than 10%; (h) subtraction of five when the frequency of reactive CD4+ or CD8+ cells is less than 3%; (i) subtraction of all points if the frequencies of reactive CD4+ or CD8+ cells are <1%. In some embodiments, based on the HLA match between the patient and the viral-specific cytotoxic T cell line, (g) and (h) are further defined as subtraction of one per integer: (a) based on the higher frequency of CD4+ cells that secrete IFN or IL-2; or (b) based on the higher frequency of CD8+ cells that secrete IFN or IL-2. In specific embodiments, determining by the processor a viral-specific cytotoxic T-cell line suitable for administering to the patient based on the cell line identification number is based at least in part on one or more of the following values for when the response to the virus is CD8-dominant: (a) product of the number of matches at HLA-A times seven; (b) product of the number of matches at HLA-DR times 6; (c) the number of matches at HLA-B; (d) the number of matches at HLA-C; (e) the number fifteen if there is a match at HLA-DR and HLA-A; (f) the number one if there is a match at HLA-B and HLA-C; (g) subtraction of one per integer when the frequency of reactive CD4+ or CD8+ cells is less than 10%; (h) subtraction of five when the frequency of reactive CD4+ or CD8+ cells is less than 3%; (i) subtraction of all points if the frequencies of reactive CD4+ or CD8+ cells are <1%. In some cases, based on the HLA match between the patient and the viral-specific cytotoxic T cell line, (g) and (h) are further defined as subtraction of one per integer: (a) based on the higher frequency of CD4+ cells that secrete IFN or IL-2; or (b) based on the higher frequency of CD8+ cells that secrete IFN or IL-2. In specific embodiments, a therapeutically effective amount of cells from the cell line having the highest cell line identification number based on the patient information is delivered to the patient.

Embodiments of the disclosure include a computer program product comprising: a non-transitory computer readable medium comprising instructions for causing an information handling system to perform the steps comprising: receiving patient information representative of one or more human leukocyte antigen (HLA) genes for a patient; and determining using an algorithm a viral-specific cytotoxic T-cell line suitable for administering to the patient based on a cell line identification number for each of a plurality of cell lines that may be selected for the patient, and based on information about the HLA gene(s) indicated in the patient information. In specific embodiments the determining step is further defined as determining a viral-specific cytotoxic T-cell line suitable for administering to the patient based on values representative of matching of HLA gene(s) of the patient with respect to corresponding HLA gene(s) in the plurality of cell lines. The information about the HLA gene(s) for the patient may comprise, for each of the two chromosomes, the identity of the HLA gene, the HLA group, and the specific HLA protein. In specific embodiments, the information of the HLA gene(s) for the patient is represented by the following expression: HLA-X*YY:ZZ, where X is an HLA gene, YY is an HLA group, and ZZ is a specific HLA protein. In some embodiments, the information of the HLA gene(s) is determined by a binary selection at YY and ZZ, said binary selection representing an allele from each of two chromosomes of the patient. In specific cases, in the formula, X represents HLA-A, HLA-B, HLA-C, or HLA-DR. In some aspects, the patient information about the HLA gene comprises information with respect to a binary selection at YY and ZZ for each of HLA-A, HLA-B, HLA-C, and HLA-DR. In specific embodiments, the determining of a viral-specific cytotoxic T-cell line suitable for administering to the patient based on the cell line identification number is further defined as identifying a T-cell line having the same HLA allele for one or both chromosomes for one or more of HLA-A, HLA-B, HLA-C, and HLA-DR. The determining of the viral-specific cytotoxic T-cell line from a plurality of cell lines that may be selected for the patient may comprise receiving information about a CD4+ or CD8+ dominant response based on information about an HLA gene for the patient. In specific aspects, when the response to the virus corresponding to the viral-specific cytotoxic T cell line is CD4 dominant, matching at HLA-DR has a weighted value for the algorithm that is greater than a weighted value for matching at HLA-B and/or a weighted value for matching at HLA-C. In some aspects, when the response to the virus corresponding to the viral-specific cytotoxic T cell line is CD4 dominant, matching at HLA-A has a weighted value for the algorithm that is greater than a weighted value for matching at HLA-B and/or a weighted value for matching at HLA-C. In some cases, when the response to the virus corresponding to the viral-specific cytotoxic T cell line is CD4 dominant, the combination of matching at one allele for one chromosome at HLA-DR and matching at one allele for one chromosome at HLA-A has a weighted value that is greater than a weighted value for the combination of matching at each allele for both chromosomes of HLA-A or for both chromosomes at HLA-DR. In particular aspects, when the response to the virus corresponding to the viral-specific cytotoxic T cell line is CD4 dominant, matching at HLA-DR in the absence of concomitant matching at HLA-A has a weighted value that is greater than a weighted value for matching at HLA-A with no concomitant matching at HLA-DR. In some aspects, when the response to the virus corresponding to the viral-specific cytotoxic T cell line is CD8 dominant, matching at HLA-DR has a weighted value for the algorithm that is greater than a weighted value for matching at HLA-B and/or a weighted value for matching at HLA-C. In specific cases, when the response to the virus corresponding to the viral-specific cytotoxic T cell line is CD8 dominant, matching at HLA-A has a weighted value for the algorithm that is greater than a weighted value for matching at HLA-B and/or a weighted value for matching at HLA-C. When the response to the virus corresponding to the viral-specific cytotoxic T cell line is CD8 dominant: the combination of (a) matching at one allele for HLA-DR and (b) matching at one allele for HLA-A may have a weighted value in the algorithm that is greater than a weighted value for (1) or (2): (1) the combination of (a) matching at both alleles of HLA-A and (b) matching at no alleles at HLA-DR, or (2) the combination of (a) matching at both alleles at HLA-DR and (b) matching at no alleles at HLA-A. In specific embodiments, when the response to the virus corresponding to the viral-specific cytotoxic T cell line is CD8 dominant, matching at HLA-DR in the absence of concomitant matching at HLA-A has a weighted value that is less than a weighted value for matching at HLA-A with no concomitant matching at HLA-DR.

In specific embodiments, the non-transitory computer readable medium is further configured to perform the instructions comprising: receiving information representative of a frequency of reactive cytotoxic T-lymphocyte (CTLs) from a plurality of cell lines that may be selected for the patient, wherein the CTLs comprise CD4+ cells and CD8+ cells and are measured by at least one of IFN secreted from CD4+ cells, IFN secreted from CD8+ cells, IL-2 secreted from CD4+ cells, and IL-2 secreted from CD8+ cells, wherein the step of determining the viral-specific cytotoxic T-cell line from a plurality of cell lines that may be selected for the patient is based on the frequency of reactive CTLs. In some cases, the non-transitory computer readable medium is further configured to perform the step comprising subjecting a plurality of cells comprising CD4+ cells and CD8+ cells, from a plurality of cell lines that may be selected for the patient, to peptides from a viral antigen and identifying a proportion of the cells that are activated by the peptides. In some cases, the determining step is further defined as determining a proportion of CD4+ cells that are activated by the peptides and determining a proportion of CD8+ cells that are activated by the peptides. In some cases, the activation is determined by measuring the level of secretion of interleukin (IL)-2 and interferon-γ (IFN) from both of the CD4+ and CD8+ cell types. In some cases, the step of determining the viral-specific cytotoxic T-cell line from a plurality of cell lines that may be selected for the patient comprises identifying among CD4+ cells and CD8+ cells the proportion of CD4+ cells that secrete IL-2 and IFN and the proportion of CD8+ cells that secrete IL-2 and IFN. In particular embodiments, when the response to the virus corresponding to the viral-specific cytotoxic T cell line is CD4 dominant, then there is a greater weighted value for the algorithm for the higher secretion level of CD4+ IFN or CD4+ IL-2. In some embodiments, determining a viral-specific cytotoxic T-cell line suitable for administering to the patient based on the cell line identification number is based at least in part on one or more of the following values for when the response to the virus is CD4-dominant: (a) the product of the number of matches at HLA-DR times seven; (b) the product of the number of matches at HLA-A times six; (c) the number of matches at HLA-B; (d) the number of matches at HLA-C; (e) the number fifteen if there is a match at HLA-DR and HLA-A; (f) the number one if there is a match at HLA-B and HLA-C; (g) subtraction of one per integer when the frequency of reactive CD4+ or CD8+ cells is less than 10%; (h) subtraction of five when the frequency of reactive CD4+ or CD8+ cells is less than 3%; and (i) subtraction of all points if the frequencies of reactive CD4+ or CD8+ cells are <1%. In some embodiments, based on the HLA match between the patient and the viral-specific cytotoxic T cell line, (g) and (h) are further defined as subtraction of one per integer: (a) based on the higher frequency of CD4+ cells that secrete IFN or IL-2; or (b) based on the higher frequency of CD8+ cells that secrete IFN or IL-2. In specific embodiments, determining by the processor a viral-specific cytotoxic T-cell line suitable for administering to the patient based on the cell line identification number is based at least in part on one or more of the following values for when the response to the virus is CD8-dominant: (a) product of the number of matches at HLA-A times seven; (b) product of the number of matches at HLA-DR times 6; (c) the number of matches at HLA-B; (d) the number of matches at HLA-C; (e) the number fifteen if there is a match at HLA-DR and HLA-A; (f) the number one if there is a match at HLA-B and HLA-C; (g) subtraction of one per integer when the frequency of reactive CD4+ or CD8+ cells is less than 10%; (h) subtraction of five when the frequency of reactive CD4+ or CD8+ cells is less than 3%; (i) subtraction of all points if the frequencies of reactive CD4+ or CD8+ cells are <1%. In some cases, based on the HLA match between the patient and the viral-specific cytotoxic T cell line, (g) and (h) are further defined as subtraction of one per integer: (a) based on the higher frequency of CD4+ cells that secrete IFN or IL-2; or (b) based on the higher frequency of CD8+ cells that secrete IFN or IL-2. In some embodiments, a therapeutically effective amount of cells from the cell line having the highest cell line identification number based on the patient information is delivered to the patient.

It is contemplated that any embodiment discussed in this specification can be implemented with respect to any method or composition of the invention, and vice versa. Furthermore, compositions of the invention can be used to achieve methods of the disclosure. For example, the method may be embedded in computer-readable medium as computer program code comprising instructions that cause a processor to perform the steps of the method. In some embodiments, the processor may be part of an apparatus with memory. In another embodiment, the method may be implemented in an algorithm such as a machine learning algorithm. The algorithm may include an input for information regarding the patient's response to the treatment, and based on the information from the patient's response, the algorithm may adjust the weights in another implementation of the algorithm. For example, the machine learning algorithm may be trained using medical data such as patient response, patient tissue type (HLA), and/or other information.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

For a more complete understanding of the disclosed methods and system, reference is now made to the following descriptions taken in conjunction with the accompanying drawings.

FIG. 1 is a flow chart illustrating a method according to some embodiments of the disclosure.

FIG. 2 is a schematic block diagram illustrating an apparatus according to some embodiments of the disclosure.

### DETAILED DESCRIPTION

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

The term “CD4-dominant” with respect to viruses as used herein refers to an immunological response to an antigen that is mostly mediated by CD4-positive T cells

The term “CD8-dominant” with respect to viruses as used herein refers to an immunological response to an antigen that is mostly mediated by CD8-positive T cells.

The terms “suitable,” “more suitable,” “better suited” and similar terms as used herein refer to a T cell line selected by one or more methods encompassed herein that has a greater chance of therapeutic efficacy and/or reduced deleterious immune response for a recipient individual compared to one or more other T cell lines not selected by one or more methods encompassed herein.

The term “virus-specific T cells” or “VSTs” or “virus-specific T cell lines” or “VST cell lines” are used interchangeably herein to refer to T cell lines, e.g., as described herein, that have been expanded and/or manufactured outside of a subject and that have specificity and potency against a virus or viruses of interest. The VSTs provided herein are third party VSTs. The VSTs may be monoclonal or oligoclonal, in embodiments. In particular embodiments the VSTs are polyclonal. As described herein, in embodiments, a viral antigen or several viral antigens are presented to naive T cells or memory T cells in peripheral blood mononuclear cells and the native CD4+ and/or CD8+ T cell populations with specificity for the viral antigens(s) expand in response.

The term “viral antigen” as used herein refers to an antigen that is protein in nature and is closely associated with a virus particle. In specific embodiments, a viral antigen is a coat protein.

As used herein, the terms “patient” or “subject” or “individual” are used interchangeably to refer to any mammal, including humans, domestic and farm animals, and zoo, sports, and pet animals, such as dogs, horses, cats, cattle, sheep, pigs, goats, rats, guinea pigs, or non-human primates, such as a monkeys, chimpanzees, baboons or rhesus. One particular mammal is a human, including adults, children, and the elderly.

The terms “treat”, “treating”, “treatment” and the like, as used herein, unless otherwise indicated, refers to reversing, alleviating, inhibiting the process of, or preventing the disease, disorder or condition to which such term applies, or one or more symptoms of such disease, disorder or condition and includes the administration of any of the compositions, pharmaceutical compositions, or dosage forms described herein, to prevent or delay the onset of the symptoms or the complications, or alleviating the symptoms or the complications, or eliminating the disease, condition, or disorder. In some instances, treatment is curative or ameliorating. In specific cases, the selected T cell line is given to an individual prior to and/or following exposure to a virus or an individual suspected of having a particular cancer or at risk for having a particular cancer (such as compared to the general population, or by having one or more risk factors).

Embodiments of the disclosure include systems, methods, and compositions that facilitate selection of a particular T cell line for therapy for an individual. In specific embodiments, the present disclosure allows for identification of a T cell line for therapy for an individual, wherein the T cell line is one of a plurality of T cell lines from which to choose, such as in a tissue bank. The present disclosure enables determination of a cell line that is better suited for a recipient individual compared to one or more other cell lines based at least on the genotype of the cells in comparison to the genotype of the recipient individual for the cells. The systems, methods, and compositions allow for selection of a T cell line that is better suited for an individual than compared to what would be selected in the absence of the systems, methods, and compositions. In specific embodiments, the T cell line is a cytotoxic T cell line, such as a viral-specific cytotoxic T cell line, although in some cases it may be a cancer antigen-specific cytotoxic T cell line. In specific embodiments, an individual is in need of therapy with cytotoxic T cells against a particular pathogenic virus or cancer antigen, and the reactivity of one or more T cell lines in a plurality of T cell lines against an antigen of the virus or cancer antigen is known or determined.

Methods include selection of an allogeneic T cell line for therapeutic administration to an individual having or suspected of having a pathogen such as a virus. In specific embodiments, the methods include selecting a T cell line allogeneic to an individual that recognizes at least one epitope of an antigen of the pathogen, wherein the method includes identification of one or a plurality of HLA alleles for the T cells, and includes identification of one or a plurality of HLA alleles for the individual in need thereof, and that also includes identification of relative activities of the T cell lines, each recognizing at least one epitope of an antigen of a virus (including the pathogenic virus for which the individual is infected or susceptible to). In specific embodiments, the method includes identification of one or a plurality of HLA alleles for the T cells, and includes identification of a corresponding one or a plurality of HLA alleles for the individual in need thereof, In specific embodiments, the systems and methods allocate a cell identification number for a particular T cell line based on a variety of parameters associated with the T cell line and in comparison to a recipient individual, with a higher cell identification number being associated with a better suited T cell line for the individual.

In particular embodiments, an individual in need of T cell therapy is an individual in need of cytotoxicity against one or more viruses. The individual may be at risk for viral infection based on a medical condition or based on a medical procedure that requires suppression of the immune system, leaving the individual at greater susceptibility to infection, in various embodiments. In specific embodiments, the individual is immunocompromised, including severely immunocompromised. In specific embodiments, the individual has undergone a transplant of any kind, such as an hematopoietic stem cell transplantation (e.g., bone marrow transplant, peripheral blood stem cell transplant, or cord blood transplant) or solid organ transplantation. The individual may be taking one or more immunosuppressive drugs (such as chemotherapy); they may have an autoimmune disease (e.g., multiple sclerosis, lupus, rheumatoid arthritis, etc.); they may have an acquired immune deficiency disorder, such as chickenpox, lupus, mono, tuberculosis, Severe Acute Respiratory Syndrome (SARS), SARS-CoV2, and/or Acquired Immune Deficiency Syndrome (AIDS) caused by the Human Immunodeficiency Virus (HIV); and so forth. The individual may be suspected of having a viral infection or known to have a viral infection and may receive the selected T cells whether or not the virus infection has been verified, such as with routine preventative medical care, including prior to, upon, and/or following transplantation. The individual may not be immunocompromised.

In embodiments, the T cell line comprises antigen specificity for at least one antigen or a portion thereof from a single virus. The virus may be BK virus (BKV), John Cunningham virus (JCV), herpesvirus, adenovirus, human immunodeficiency virus, influenza virus, ebola virus, herpesvirus-6 (HHV-6), human herpesvirus-8 (HHV-8), poxvirus, rhabdovirus, paramyxovirus, cytomegalovirus (CMV), Epstein Barr virus (EBV), respiratory syncytial virus (RSV), parainfluenza (PIV), rhinovirus, human metapneumovirus (hMPV), Hepatitis B virus (HBV), Hepatitis C virus (HCV), Bocavirus, Coronavirus (e.g., SARS or SARS-CoV-2), Lymphocytic choriomeningitis virus (LCMV), Mumps, Measles, Parvovirus B, Rotavirus, merkel cell virus, herpes simplex virus, human papillomavirus (HPV), Human immunodeficiency virus (HIV), Human T-cell leukemia virus type 1 (HTLV1), West Nile Virus, zika virus, and so forth.

The T cells may be directed to a specific antigen of a virus. In some embodiments, the virus is cytomegalovirus (CMV), with pp65 or IE1 being examples of antigens. The virus may be Epstein-Barr virus (EBV), in which case the antigen may be EBNA1, EBNA2, EBNA3A, EBNA3B, EBNA3C, LMP1, or LMP2. In certain embodiments, the virus is influenza, and the Influenza antigens may be selected from influenza A antigens NP1, MP1, and a combination thereof. In some cases, the virus is RSV, and the RSV antigens may be selected from N, F, and a combination thereof. In certain aspects, the virus is hMPV, and the hMPV antigens may be selected from F, N, M2-1, M, and a combination thereof. In particular cases, the virus is PIV, and the PIV antigens may be selected from M, HN, N, F, and a combination thereof. In embodiments, the virus is EBV, and the EBV antigens may be selected from FMP2, EBNA1, BZFF1, and a combination thereof. In specific embodiments, the virus is adenovirus, and the adenovirus antigens are selected from Hexon, Penton, and a combination thereof. The virus may be the BK virus, and the BK virus antigens may be selected from VP1, large T, and a combination thereof. In cases wherein the virus is HHV6, the HHV6 antigens may be selected from U90, U11, U14, and a combination thereof. In cases wherein the virus is HHV8, antigens from HHV8 may be selected from LANA-1 (ORF3); LANA-2 (vIRF3, K10.5); vCYC (ORF72); RTA (ORF50); vFLIP (ORF71); Kaposin (ORF12, K12); gB (ORF8); MIR1 (K3); SSB (ORF6); TS(ORF70), and a combination thereof. In embodiments wherein the virus is HBV, the antigens from HBV may be selected from HBV core antigen, HBV Surface Antigen, and a combination of HBV core antigen and HBV Surface Antigen.

### I. HLA Matching and Cell Reactive Frequency Determination

In this disclosure, the determination of a suitable T cell line for an individual is based on the HLA typing of both (a) the particular cell lines that are available, and (2) an intended recipient individual. Specifically, one or more HLA alleles for a given individual in need of cell therapy is compared to one or more HLA alleles of one or more cell lines in a grouping or bank of cell lines. The cell line having the greatest matching between the HLA alleles may be selected for therapy for the individual, although in specific cases other parameters than only HLA allele matching are additionally considered. In any case, a measurement of suitability (that may be considered efficacy and/or safety upon administration) between a given cell line and a recipient individual is represented in a cell identification number that includes values of multiple scores for a variety of parameters including matching at one or more HLA alleles and the level of cell reactive frequencies for a given T cell line, in specific embodiments.

The skilled artisan recognizes the conventional format of given HLA alleles, as illustrated below.

Each HLA gene has an allele corresponding to each pair of chromosomes. Therefore, in an algorithm that includes information about HLA alleles, for a given HLA gene (e.g., HLA-A, HLA-B, HLA-DR, or HLA C) there is a line that represents Field 1 and Field 2 for each chromosome. For example, in the following examples of code lines for an algorithm,
cel_A_1_a: Cell line HLA gene A field 1 allele A is represented by 02 above
cel_A_2_a: Cell line HLA gene A field 2 allele A is represented by 01 above, and so forth.

As further elaboration, information about an HLA allele may be represented by the following expression:

HLA-X*YY:ZZ
where X is an HLA gene (in one example, X represents HLA-A, HLA-B, HLA-C, or HLA-DR), YY is an HLA group, and ZZ is a specific HLA protein. Thus, YY:ZZ is representative of a specific allele of an HLA gene. In specific embodiments of the method, the information of the HLA gene(s) is determined by a binary selection at YY and ZZ, representing an allele from each of two chromosomes of a recipient individual.

In some embodiments, the allele of one or more given HLA genes is the same between the T cell line and the individual in need of the cells, whereas in other embodiments the allele of one or more given HLA genes is different between the T cell line and the individual in need of the cells. For a comparison between a given HLA allele of the cells and a corresponding HLA allele of an individual, more points towards a cell identification number are valued in the number when there is a match at the allele, and fewer points towards a cell identification number are valued when there is not a match.

In specific embodiments, a T cell line is selected based at least in part on HLA matching between the cells of the T cell line and an individual in need of the T cell line for therapy, and methods and compositions herein utilize mathematical formulas that identify such an optimized compatibility. These mathematical formulas encompass a variety of parameters in considering suitability of a specific T cell line for an individual. The disclosed processes herein eliminate the need to experimentally qualify a T cell line prior to use for treatment. Instead, identification of accurate tissue typing (in the form of HLA typing) at least in part indicates a most suitable cell line among a plurality of cell lines for an individual in need and based on HLA genotype.

In specific embodiments, with respect to an individual in need of treatment with a cell line, there are a certain number of points allotted to each cell line based on the degree of HLA matching between one of multiple cell lines from which to choose and the individual in need. A plurality of cell lines are analyzed for suitability for the individual and based on their HLA genotype, and in some embodiments, in addition to HLA genotype, the frequency of reactive T cells is also considered in the identification of a suitable or most suitable cell line among a plurality of cell lines. In particular embodiments, the HLA matching also identifies whether or not there is a CD4-dominant response or a CD8-dominant response, and this information may also be considered in the identification of a suitable or most suitable cell line among a plurality of cell lines. In particular embodiments, the algorithm determines whether the response is going to be CD4-dominant or CD8-dominant based on the HLA matching and then considers the CD4 frequencies or the CD8 frequencies of the T cells for a given cell line. In addition, in particular embodiments the CD4/CD8 dominance depends on the virus in addition to the matching between the cell line and the individual in need thereof. In specific examples, SARS-CoV-2, BKV, and JCV are CD4 dominant, and CMV is CD8 dominant. A virus may be determined to be CD4-dominant vs. CD8-dominant by stimulating T-cells with viral antigens and measuring the frequencies of IFN, IL-2, etc., in CD4 and CD8 T-cells. For some viruses, the cytokines are mostly secreted by the CD4+ cells, and for other viruses the cytokines are mostly secreted by CD8+ cells. Also, one can measure phenotype changes in the T-cells after exposure to viral antigens, such as by flow cytometry or other techniques.

In particular embodiments, the type of virus determines a weight value of the match in the algorithm. For example, for viruses that elicit a CD4-dominant response (e.g., BKV, adenoviruses) the matching at HLA-DR is given a higher weighted value than matching at HLA-A. For CD8 viruses, the matching at HLA-A is given a higher weighted value than matching at HLA-DR.

In specific embodiments for a virus that elicits a CD4-dominant response, the frequencies of CD4 cells producing IFN and IL2 are considered particularly when there is an HLA match at HLA-DR and no corresponding match at HLA-A; in such cases, no weight may be given to CD8 frequencies. However, if there is a match at HLA-A and no match at HLA-DR, then in specific embodiments the frequencies of CD8 cells producing interferon and IL2 are considered and no weight may be given to CD4 frequencies.

The determination of reactivity of the T cells may be performed in any suitable manner, but in specific cases a group of cells including CD4 and CD8 cells are stimulated with peptides from a viral antigen. In some cases, the peptides are of a length of 3-100 amino acids in length and they may or may not overlap the entire antigen. The peptides may be of a length of about 3-100, 3-75, 3-50, 3-25, 5-100, 5-75, 5-50, 5-25, 10-100, 10-75, 10-50, 10-25, 20-100, 20-75, 20-50, 30-100, 30-75, 30-50, 40-100, 40-75, 40-50, 50-100, 50-75, 75-100, or any range derivable therein. In specific cases, a library of peptides includes peptides overlapping one or more viral antigens and may be of the length ranges noted above; the peptides may overlap totally or partially one or more viral antigens. The extent to which the CD4 and CD8 cells secrete IFN and IL-2 are correlative with reactivity of the cells, such that higher levels of their secretion indicate the greater reactivity of the cells. In particular embodiments, the proportion of the level of secretion of IFN and IL-2 for each of CD4 and CD8 is determined and is included in the calculations. The calculation of the reactive frequencies may or may not be determined for one or more particular cell lines ahead of their storage in a bank. That is, for one or more viral antigens, the reactive frequencies in response to the antigen may be determined ahead of the use of the cells and ahead of need from a specific individual.

In the mathematical calculations with respect to reactive frequencies, the calculation for a particular cell line may include one point per each integer when the frequency of reactive CTLs is below 10 (and no loss of points if the frequency is 10 or greater, and all loss of points if the frequency is lower than 1 (that is, the algorithm gives that line a score of 0 and the line is not selected)). In one example in which:
10% of CD4+ cells secrete IFN (−0)
3% of CD4+ cells secrete IL-2 (−7)
7% of CD8+ cells secrete IFN (−3)
5% of CD8+ cells secrete IL-2 (−5),
in comparison to the value 10, there could be zero points subtracted for the CD4+ cells that secrete IFN, seven points subtracted for CD4+ cells that secrete IL-2, three points subtracted for CD8+ cells that secrete IFN, and five points subtracted for CD8+ cells that secrete IL-2. However, the HLA matching of the CTLs and the patients are taken into consideration when including calculations of the frequencies for the points for the cell line. That is, depending on the HLA of the CTLs and patients, one can utilize either the CD4 frequency or the CD8 frequency in the calculation, and within the CD4 IFN/IL-2 values or CD8 IFN/IL-2 values, the larger number is selected. Therefore, in the example above, depending on the HLA value, the cell line points would either have 0 points subtracted or three points subtracted. If there is a match at HLA-DR and not a match at HLA-A (or a match at both), then the CD4 values are utilized in the algorithm (so, 0 points subtracted), and if there is a match at HLA-A and not a match at HLA-DR, then the CD8 values are utilized in the algorithm (so, three points are subtracted).

In another example, a value is given to the HLA matching of 36 and there is a match at HLA-A and at HLA-DR, In such a case, CD4 frequencies are reflected in the algorithm, irrespective of the value of the CD8 frequencies. In this example, the CD4 IL2 is 7% and the CD4 IFN is 5%. So, the highest is 7%. Then, 10−7=3, so the score is 36−3=33

Another rule for the algorithm is that if the highest frequency is less than three, than an additional five points are subtracted from the value. Thus, in another example for when the value of 36 is given to the HLA matching, if the highest frequency between IFN or IL-2 (for either CD4 or CD8) was 2, then:
10−2=8, but because the highest frequency is less than 3, an additional 5 points are subtracted: Score is 36−8−5=23

In particular embodiments, selection of a T cell line includes information about a CD4+ or CD8+ dominant response to the virus in addition to information about an HLA gene for the patient.

In particular embodiments, the virus type (for example BKV) determines at least in part a weight for HLA matching. In the case of BKV, as an example of a CD4-dominant virus, more points are allotted for a HLA-DR match than for an HLA-A match. Then, the match determines whether the CD4 frequencies or the CD8 frequencies are utilized. For example, for BKV CTLs one seeks a line with HLA-DR matches (or HLA-DR+HLA-A that is even more desired), and then the CD4 frequencies are utilized. On the other hand, for BKV CTLs if the best line that can be found only has matches at HLA-A (and not HLA-DR), then that line can be utilized and the CD8 frequencies are taken into consideration instead of the CD4 frequencies.

In some embodiments, when response to the virus corresponding to the viral-specific cytotoxic T cell line is CD4 dominant, then there is a greater weighted value for the algorithm for the higher secretion level of CD4+ IFN or CD4+ IL-2. In some embodiments, when the response to the virus corresponding to the viral-specific cytotoxic T cell line is CD8 dominant, then there is a greater weighted value for the algorithm for the higher secretion level of CD8+ IFN or CD8+ IL-2. However, in at least some cases the state of HLA matching between the T cell line and an individual in need of the cells determines a greater weighted value in the algorithm for CD8 frequencies over CD4 frequencies, or vice versa. For example, if the HLA match between a T cell line and an individual in need of cells is at HLA-A (with no match at HLA-DR) then there is a greater weighted value for CD8 frequencies. In at least some cases when the virus corresponding to the viral-specific cytotoxic T cell line is CD4 dominant, matching at HLA-DR has a weighted value for the algorithm that is greater than a weighted value for matching at HLA-B and/or a weighted value for matching at HLA-C. In certain cases, when the response to the virus corresponding to the viral-specific cytotoxic T cell line is CD4 dominant, matching at HLA-A has a weighted value for the algorithm that is greater than a weighted value for matching at HLA-B and/or a weighted value for matching at HLA-C. In specific embodiments, when the response to the virus corresponding to the viral-specific cytotoxic T cell line is CD4 dominant, the combination of matching at one allele for one chromosome at HLA-DR and matching at one allele for one chromosome at HLA-A has a weighted value that is greater than a weighted value for the combination of matching at each allele for both chromosomes of HLA-A or for both chromosomes at HLA-DR. In specific embodiments, when the response to the virus corresponding to the viral-specific cytotoxic T cell line is CD4 dominant, matching at HLA-DR in the absence of concomitant matching at HLA-A has a weighted value that is greater than a weighted value for matching at HLA-A with no concomitant matching at HLA-DR.

In certain embodiments, when the response to the virus corresponding to the viral-specific cytotoxic T cell line is CD8 dominant, matching at HLA-DR has a weighted value for the algorithm that is greater than a weighted value for matching at HLA-B and/or a weighted value for matching at HLA-C. In some cases, when the response to the virus corresponding to the viral-specific cytotoxic T cell line is CD8 dominant, matching at HLA-A has a weighted value for the algorithm that is greater than a weighted value for matching at HLA-B and/or a weighted value for matching at HLA-C. When the virus corresponding to the viral-specific cytotoxic T cell line is CD8 dominant:
the combination of (a) matching at one allele for HLA-DR and (b) matching at one allele for HLA-A has a weighted value in the algorithm that is greater than a weighted value for (1) or (2):
(1) the combination of (a) matching at both alleles of HLA-A and (b) matching at no alleles at HLA-DR, or
(2) the combination of (a) matching at both alleles at HLA-DR and (b) matching at no alleles at HLA-A, in some cases.

In some embodiments, when the response to the virus corresponding to the viral-specific cytotoxic T cell line is CD8 dominant, matching at HLA-DR in the absence of concomitant matching at HLA-A has a weighted value that is less than a weighted value for matching at HLA-A with no concomitant matching at HLA-DR.

In particular embodiments, an algorithm selects a T cell line for an individual in need thereof based on a cell line identification number corresponding to the particular T cell line. A variety of factors may be included in the weighted analysis towards the cell line identification number for a particular T cell line and as represented by the cell line identification number. In specific embodiments, the cell line identification number is based at least in part on one or more of the following values for when the response to the virus is CD4-dominant:
(a) the product of the number of matches at HLA-DR times seven;
(b) the product of the number of matches at HLA-A times six;
(c) the number of matches at HLA-B;
(d) the number of matches at HLA-C;
(e) the number fifteen if there is a match at HLA-DR and HLA-A;
(f) the number one if there is a match at HLA-B and HLA-C;
(g) subtraction of one per integer when the frequency of reactive CD4+ or CD8+ cells is less than ten;
(h) subtraction of five per integer when the frequency of reactive CD4+ or CD8+ cells is less than three; and
(i) subtraction of all points if the frequencies of reactive CD4+ or CD8+ cells are <1%.

In specific embodiments, the cell line identification number is based at least in part on one or more of the following values for when the response to the virus is CD8-dominant:
(a) product of the number of matches at HLA-A times seven;
(b) product of the number of matches at HLA-DR times 6;
(c) the number of matches at HLA-B;
(d) the number of matches at HLA-C;
(e) the number fifteen if there is a match at HLA-DR and HLA-A;
(f) the number one if there is a match at HLA-B and HLA-C;
(g) subtraction of one per integer when the frequency of reactive CD4+ or CD8+ cells is less than 10%;
(h) subtraction of five when the frequency of reactive CD4+ or CD8+ cells is less than 3%;
(i) subtraction of all points if the frequencies of reactive CD4+ or CD8+ cells are <1%.

As indicated in (a) above for CD4-dominant viral responses, as part of the cell line identification number, one may consider the value of the product of the number of matches at HLA-DR times seven. The number of matches at a given locus may be 0, 1, 1.5 (for when the cell line is homozygous), or 2. An example of such scoring is provided below:

| Patient | CTL | Value |
|---|---|---|
| 02:01 03:02 | 04:01 05:01 | 0 |
| 02:01 03:02 | 02:01 05:01 | 1 |
| 02:01 03:02 | 02:01 03:02 | 2 |
| 02:01 02:01 | 02:01 05:01 | 1 |
| 02:01 03:01 | 02:01 02:01 | 1.5 |
| 02:01 02:01 | 02:01 02:01 | 1.5 |

In the example above, in the first line a value of 0 is given to the cell identification number because none of the alleles are matching between the patient and CTLs; in the second line a value of 1 is given because the patient and CTLs match at 02:01 but not at 03:02/05:01; in the third line a value of 2 is given because the patient and CTLs match at both 02:01 and 03:02; in the fourth line a value of 1 is given because only one TCR in the CTLs (02:01) interacts, irrespective of the patient being homozygous. In the fifth line, a value of 1.5 is given because both TCRs interact (so it could be 2 points), but the CTLs are homozygous; because both TCRs are identical in this example and in the sixth line, it is considered that although both TCRs interact with the target cells, the situation is not as preferable as when both TCRs interact but they are different to each other; therefore it is given 1.5 points. The sixth line a value of 1.5 is given analogously. In sum, the rule is that one point is given per match between patient and cell line except when the cell line is homozygous, and then it gets 1.5 points provided there is at least one match.

### II. Therapeutic Applications of Selected T Cell Lines

The disclosure encompasses methods of treating an individual that has or is at risk for or is suspected of having a virus, comprising: (a) selecting an allogeneic T cell line for therapeutic administration to the patient according to any of the methods encompassed herein; and (b) administering a therapeutically effective amount of a population of T cells derived from the selected allogeneic T cell line to the individual. The cells may be given to the individual by any suitable manner, but in specific embodiments a population of T cells derived from the selected allogeneic T cell line are administered to the individual by infusion in the blood or in tissue or in a body cavity, or administered intrathecally or by inhalation, for example. In specific embodiments, the infusion is a bolus intravenous infusion of a population of T cells derived from the selected allogeneic T cell line.

The amount to be administered can be determined based on the condition of the individual. In specific embodiments, the administration includes at least about 1×10⁵ T cells/kg/dose/week to the individual, wherein the population of T cells is derived from the selected allogeneic T cell line. In some embodiments, the administering comprises administering about 1×10³ to 1×10¹⁰ T cells/kg/dose/week to the individual, including any range derivable therein, wherein the population of T cells is derived from the selected allogeneic T cell line. In some embodiments, the administering comprises administering at least about 1×10⁵ cells/kg/dose/week to the individual, wherein the population of T cells is derived from the selected allogeneic T cell line. In some embodiments, the administering comprises administering at least about 2×10⁶ T cells/kg/dose/week to the individual, wherein the population of T cells is derived from the selected allogeneic T cell line.

In certain embodiments, the above-described dosage regimens are carried out for at least 1, 2, 3, 4, 5, 6, 7, 8, 9, 10, 11, 12, or more weeks, such that at least 2, 3, 4, 5, 6, 7, 8, 9, 10, 11, or 12 doses are administered, respectively.

In some embodiments, suitable doses for a therapeutic effect would be at least 10⁵ or between about 10⁵ and about 10¹⁰ cells per dose, for example, such as in a series of dosing cycles. An exemplary dosing regimen consists of four one-week dosing cycles of escalating doses, starting at least at about 10⁵ cells on Day 0, for example increasing incrementally up to a target dose of about 10¹⁰ cells within several weeks of initiating an intra-patient dose escalation scheme.

### III. System and Algorithm for Selection of Viral-Specific Cytotoxic T-Cell Lines for the Management of Therapy for Patients

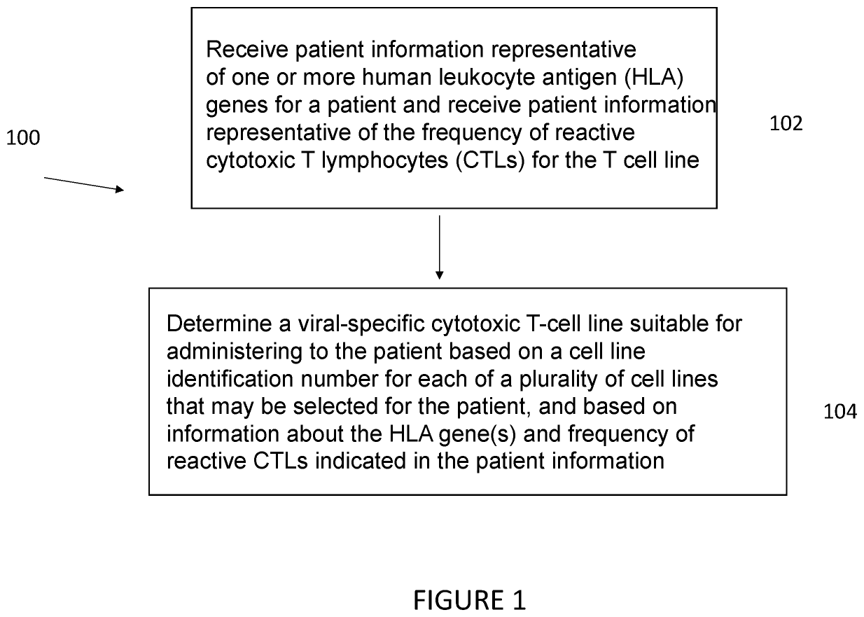

FIG. 1 illustrates a method 100 for selecting an allogeneic T cell line for therapeutic administration to an individual having or suspect of having a pathogen such as a virus. At block 102, a processor may receive patient information representative of one or more human leukocyte antigen (HLA) genes for a patient and also receive information regarding frequency of reactive CTLs for a T cell line. The processor using an algorithm may determine a viral-specific cytotoxic T-cell line suitable for administering to the patient based on a cell line identification number for each of a plurality of cell lines that may be selected for the patient, and based on information about the HLA gene(s) indicated in the patient information at block 104. For example, the processor may implement a non-linear regression model, linear regression model, or another type of algorithm to identify treatment. In some embodiments, the processor may be included in an apparatus with memory and/or another component.

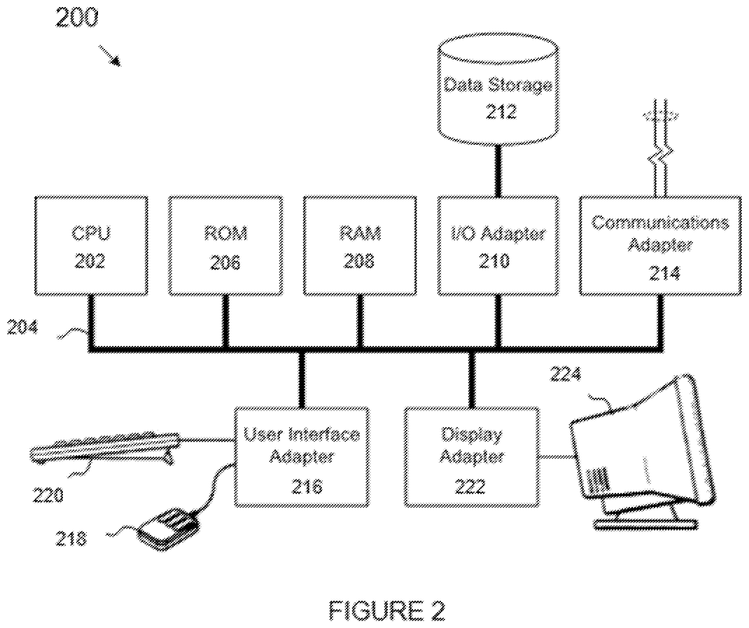

FIG. 2 illustrates the apparatus 200 that may implement the disclosed methods. For example, the processor may be a central processing unit (CPU) 202. The CPU 202 may receive patient information representative of one or more human leukocyte antigen (HLA) genes for a patient. In some embodiments, the processor may be a general-purpose CPU, a graphics processing unit (GPU), microprocessor, a processing unit, or the like. The CPU 202 may support the algorithm for selecting an allogeneic T cell line for therapeutic administration to the patient, such as the method or logical operations, according to present disclosure. The apparatus 200 may store the algorithm in the data storage 212, Random Access Memory (RAM) 208, and/or other memory. For example, the system 200 may store the algorithm and/or various data structures used by the algorithm in a SRAM, DRAM, SDRAM, or the like for implementation. In some embodiments, the data storage 212 may include a hard drive, a Compact Disk (CD) drive, a floppy disk drive, or a tape drive.

In another embodiment, the system 200 may also store the patient information representative of one or more human leukocyte antigen (HLA) genes for a patient in the data storage 212, Random Access Memory (RAM) 208, and/or other memory. The data storage 212 may be connected to an input/output (I/O) adapter 210, which is connected to the apparatus bus 214. Apparatus 200 may also include a Read Only Memory (ROM) 206 which may be PROM, EPROM, EEPROM, optical storage, or the like. The ROM 206 may also store information such as the patient information for the apparatus 200.

The apparatus bus 214 may connect the CPU 202 to the I/O adapter 210, a communications adapter 214, a user interface adapter 216, and a display adapter 222. In some embodiments, the communications adapter 214 may connect the apparatus 200 to a network. For example, the communications adapter 214 may connect the apparatus 200 with a wired or wireless connection to a server. The server may include a handheld device or another device to process medical data. For example, the server may be a remote tablet in another hospital or computer in another geographic region. Before the CPU 202 in the apparatus 200 receives the information to implement the disclosed methods, the patient information may be stored on a remote server or device. In some embodiments, the information may be received through a LAN, WAN, and/or Internet connection that may have a firewall and/or network boundary.

The apparatus 200 with the user interface adapter 216 may provide user interfaces. Users may interact with the apparatus 200 using an user interface, such as a keyboard 220 or a mouse 218, and may view the selected allogeneic T cell line and/or other data on the display 224. In certain embodiments, the display 224 may display a graphical user interface related to the disclosed methods.

In specific embodiments, a database with the information regarding the CTL cell lines contains the following variables:

### ID: Cell Line Identification Number

cel_A_1_a: Cell line HLA gene A field 1 allele A
cel_A_2_a: Cell line HLA gene A field 2 allele A
cel_A_1_b: Cell line HLA gene A field 1 allele B
cel_A_2_b: Cell line HLA gene A field 2 allele B
cel_B_1_a: Cell line HLA gene B field 1 allele A
cel_B_2_a: Cell line HLA gene B field 2 allele A
cel_B_1_b: Cell line HLA gene B field 1 allele B
cel_B_2_b: Cell line HLA gene B field 2 allele B
cel_C_1_a: Cell line HLA gene C field 1 allele A
cel_C_2_a: Cell line HLA gene C field 2 allele A
cel_C_1_b: Cell line HLA gene C field 1 allele B
cel_C_2_b: Cell line HLA gene C field 2 allele B
cel_DR_1_a: Cell line HLA gene DRβ1 field 1 allele A
cel_DR_2_a: Cell line HLA gene DRβ1 field 2 allele A
cel_DR_1_b: Cell line HLA gene DRβ1 field 1 allele B
cel_DR_2_b: Cell line HLA gene DRβ1 field 2 allele B
CD4_IFN: Frequencies of Interferon-γ CD4 T-cells
CD4_IL2: Frequencies of Interleukin-2 CD4 T-cells
CD8_INF: Frequencies of Interferon-γ CD8 T-cells
CD8_IL2: Frequencies of Interleukin-2 CD4 T-cells

The following variables may be entered and may correspond to the HLA of the patient
pa_A_1_a: Cell line HLA gene A field 1 allele A
pa_A_2_a: Cell line HLA gene A field 2 allele A
pa_A_1_b: Cell line HLA gene A field 1 allele B
pa_A_2_b: Cell line HLA gene A field 2 allele B
pa_B_1_a: Cell line HLA gene B field 1 allele A
pa_B_2_a: Cell line HLA gene B field 2 allele A
pa_B_1_b: Cell line HLA gene B field 1 allele B
pa_B_2_b: Cell line HLA gene B field 2 allele B
pa_C_1_a: Cell line HLA gene C field 1 allele A
pa_C_2_a: Cell line HLA gene C field 2 allele A
pa_C_1_b: Cell line HLA gene C field 1 allele B
pa_C_2_b: Cell line HLA gene C field 2 allele B
pa_DR_1_a: Cell line HLA gene DRβ1 field 1 allele A
pa_DR_2_a: Cell line HLA gene DRβ1 field 2 allele A
pa_DR_1_b: Cell line HLA gene DRβ1 field 1 allele B
pa_DR_2_b: Cell line HLA gene DRβ1 field 2 allele B

One example of an algorithm coding in SPSS language is provided below. The same algorithm can be coded on other languages depending on the IT system desired.

recode cel_c_1_a cel_c_2_a cel_c_1_b cel_c_2_b (sysmis=0)(else=copy).
do if (cel_a_1_a =pa_a_1_a and cel_a_2_a= pa_a_2_a).
compute a1=1.
else.
compute a1=0.
end if.
do if (cel_a_1_a =pa_a_1_b and cel_a_1_a =pa_a_1_b).
compute b1=1.
else.
compute b1=0.
end if.
do if (cel_a_1_b =pa_a_1_a and cel_a_2_b = pa_a_2_a).
compute c1=1.
else.
compute c1=0.
end if.
do if (cel_a_1_b =pa_a_1_b and cel_a_2_b= pa_a_2_b).
compute d1=1.
else.
compute d1=0.
end if.
do if (cel_a_1_a =cel_a_1_b and cel_a_2_a= cel_a_2_b).
compute h1=1.
else.
compute h1=0.
end if.
do if (a1=1 or b1=1).
compute e1=1.
else.
compute e1=0.
end if.
do if (c1=1 or d1 =1).
compute f1=1.
else.
compute f1=0.
end if.
compute a= e1 + f1.
if (h1=1 and a=2) a=1.5.
execute.
delete variables a1 b1 c1 d1 e1 f1 h1.
do if (cel_b_1_a =pa_b_1_a and cel_b_2_a= pa_b_2_a).
compute a1=1.
else.
compute a1=0.
end if.
do if (cel_b_1_a =pa_b_1_b and cel_b_1_a =pa_b_1_b).
compute b1=1.
else.
compute b1=0.
end if.
do if (cel_b_1_b =pa_b_1_a and cel_b_2_b = pa_b_2_a).
compute c1=1.
else.
compute c1=0.
end if.
do if (cel_b_1_b =pa_b_1_b and cel_b_2_b= pa_b_2_b).
compute d1=1.
else.
compute d1=0.
end if.
do if (cel_b_1_a =cel_b_1_b and cel_b_2_a= cel_b_2_b).
compute h1=1.
else.
compute h1=0.
end if.
do if (a1=1 or b1=1).
compute e1=1.
else.
compute e1=0.
end if.
do if (c1=1 or d1 =1).
compute f1=1.
else.
compute f1=0.
end if.
compute b= e1 + f1.
if (h1=1 and b=2) b=1.5.
execute.
delete variables a1 b1 c1 d1 e1 f1 h1.
do if (cel_c_1_a =pa_c_1_a and cel_c_2_a= pa_c_2_a).
compute a1=1.
else.
compute a1=0.
end if.
do if (cel_c_1_a =pa_c_1_b and cel_c_1_a =pa_c_1_b).
compute b1=1.
else.
compute b1=0.
end if.
do if (cel_c_1_b =pa_c_1_a and cel_c_2_b = pa_c_2_a).
compute c1=1.
else.
compute c1=0.
end if.
do if (cel_c_1_b =pa_c_1_b and cel_c_2_b= pa_c_2_b).
compute d1=1.
else.
compute d1=0.
end if.
do if (cel_c_1_a =cel_c_1_b and cel_c_2_a= cel_c_2_b).
compute h1=1.
else.
compute h1=0.
end if.
do if (a1=1 or b1=1).
compute e1=1.
else.
compute e1=0.
end if.
do if (c1=1 or d1 =1).
compute f1=1.
else.
compute f1=0.
end if.
compute c= e1 + f1.
execute.
if (h1=1 and c=2) c=1.5.
execute.
delete variables a1 b1 c1 d1 e1 f1 h1.
do if (cel_dr_1_a =pa_dr_1_a and cel_dr_2_a= pa_dr_2_a).
compute a1=1.
else.
compute a1=0.
end if.
do if (cel_dr_1_a =pa_dr_1_b and cel_dr_1_a =pa_dr_1_b).
compute b1=1.
else.
compute b1=0.
end if.
do if (cel_dr_1_b =pa_dr_1_a and cel_dr_2_b = pa_dr_2_a).
compute c1=1.
else.
compute c1=0.
end if.
do if (cel_dr_1_b =pa_dr_1_b and cel_dr_2_b= pa_dr_2_b).
compute d1=1.
else.
compute d1=0.
end if.
do if (cel_dr_1_a =cel_dr_1_b and cel_dr_2_a= cel_dr_2_b).
compute h1=1.
else.
compute h1=0.
end if.
do if (a1=1 or b1=1).
compute e1=1.
else.
compute e1=0.
end if.
do if (c1=1 or d1 =1).
compute f1=1.
else.
compute f1=0.
end if.
compute dr= e1 + f1.
if (h1=1 and dr=2) dr=1.5.
execute.
delete variables a1 b1 c1 d1 e1 f1 h1.
if (dr>0 and a>0) tier=1.
if (dr>0 and a=0) tier=2.
if (dr=0 and a>0) tier=2.
if (dr=0 and a=0) tier=3.
if (dr=2 and a=0) #res=2.
if (dr=1.5 and a=0) #res=2.
if (dr=1 and a=0) #res=2.
if (dr=2 and a=1.5) #res=1.
if (dr=2 and a=1) #res=1.
if (dr=1.5 and a=1) #res=1.
if (dr=2 and a=2) #res=0.
if (dr=1.5 and a=1.5) #res=0.
if (dr=1 and a=1) #res=0.
if (dr=0 and a=2) #res=−2.
if (dr=0 and a=1.5) #res=−2.
if (dr=0 and a=1) #res=−2.
if (dr=1.5 and a=2) #res=−1.
if (dr=1 and a=2) #res=−1.
if (dr=1 and a=1.5) #res=−1.
if (#res= 2 or #res=1 or #res=0 or #res=−1) freq= max (cd4_ifn, cd4_il2).
if (#res= −2) freq= max (cd8_inf, cd8_il2).
compute freq=rnd(freq).
do if (a<>0 and dr<>0).
compute #adr=1.
else.
compute #adr=0.
end if.
do if (b<>0 and c<>0).
compute #bc1=1.
else.
compute #bc1=0.
end if.
recode freq (low thru 10=copy)(else=10)(sysmis=sysmis) into fre1.
recode freq (low thru 3=5)(else=0)(sysmis=sysmis) into fre2.
For CD4-dominant viral responses, compute sc= dr*7 +a*6 +#adr*15 + b + c + #bc1.
compute score= sc -(10-fre1) -fre2.
if (freq<1) score=0.
if (score<0) score=0.
if (tier=3) score=0.
For CD8-dominant viral responses, compute sc= dr*6 +a*7 +#adr*15 + b + c + #bc1
compute score= sc -(10-fre1) -fre2.
if (freq<1) score=0.
if (score<0) score=0.
if (tier=3) score=0.
Then,
sort cases by score(d) freq(d).
list id score tier/cases to 3.

The output may comprise the ID number and other information of the best CTL lines (e.g., best 2, 3, 4, 5, and so on) in descending order (best to worse), for example.

The following examples are included to demonstrate particular embodiments of the disclosure. In certain embodiments, the algorithm may be a non-linear regression model, linear regression model, or another type of algorithm. For example, the algorithm may be implemented as a machine learning algorithm.

Machine learning models may include logistic regression techniques, linear discriminant analysis, linear regression analysis, artificial neural networks, machine learning classifier algorithms, or classification/regression trees in some embodiments. In some aspects, the machine learning may include one or more artificial neural networks, which may include an interconnected group of artificial neurons (e.g., neuron models) for modeling relationships between parameters, such as human leukocyte antigen (HLA) genes for a patient and a viral-specific cytotoxic T-cell line suitable for administering to the patient. In some aspects, the machine learning may include one or more convolutional neural networks, which are a type of feed-forward artificial neural network. Convolutional neural networks may include collections of neurons that each have a receptive field and that collectively tile an input space. In some aspects, the machine learning may include one or more deep learning architectures, such as deep belief networks and deep convolutional networks, which are layered neural networks architectures in which the output of a first layer of neurons becomes an input to a second layer of neurons, the output of a second layer of neurons becomes and input to a third layer of neurons, and so on. Deep neural networks may be trained to recognize a hierarchy of features. In various aspects, machine learning systems may employ Naive Bayes predictive modeling analysis of several varieties, learning vector quantization, or implementation of boosting algorithms such as Adaboost or stochastic gradient boosting systems for iteratively updating weighting to train a machine learning classifier to determine a relationship between an influencing attribute, such as HLA genes for a patient, and a treatment with a cell line and/or a degree to which such an influencing attribute affects the outcome of such a system or a treatment with a cell line.

### EXAMPLES

The following examples are included to demonstrate certain non-limiting aspects of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention.

### Example 1

### Examples of Clinical Application of Methodology for Selection of Viral-Specific Cytotoxic T-Cell Lines for the Management of Patients With Viral Infections

The present example concerns selection of suitable viral-specific T cell lines for patients in need thereof.

### BKV CTL for Hemorrhagic Cystitis

A clinical trial was performed (NCT02479698) to assess the feasibility, safety, and efficacy of administering most closely human leukocyte antigen (HLA)-matched third-party BKV-specific cytotoxic T lymphocytes (CTLs), generated from 26 healthy donors and banked for off-the-shelf use (Olson et al, Journal of Clinical Oncology 2021 39:24, 2710-2719). Briefly, a method was developed to select the optimal cytotoxic T-cell line to be administered to a given patient by calculating a score that takes into consideration several factors such as the specific HLA matches between the cell line and the patient, as well as measurements of the potency of the cell line and then choosing for each patient the cell line with the highest score. Fifty-nine patients were treated who developed BKV-HC following allogenic hematopoietic stem cell transplantation (AHSCT). Response to BKV CTL infusion was rapid; the day 14 overall response rate was 67.7% (40 of 59 evaluable patients), which increased to 81.6% among evaluable patients at day 45 (40 of 49 evaluable patients. No patient lost a previously achieved response. There were no cases of de novo grade 3 or 4 graft-versus-host disease (GVHD), graft failure or infusion-related toxicities. BKV-CTLs were identified in patient blood samples up to 3 months post-infusion and their in vivo expansion predicted for clinical response. A matched-pair analysis revealed that, compared to standard of care, after accounting for prognostic covariate effects, treatment with BKV-CTLs resulted in higher probabilities of response at all follow-up timepoints as well as significantly lower transfusion requirement.

Twenty-seven additional patients were enrolled (total 86), analyzed the data was analyzed in order to identify prognostic factors for response. The 64 patients who had received a single CTL infusion were focused on. Table 1 shows the patients characteristics.

TABLE 1
Characteristics of 64 hemorrhagic cystitis patients who
received a single CTLs infusion
Parameter

| Age at Transplant | |
|---|---|
| Median | 47 |
| (Range) | (16-70) |
| Gender, n (%) | |
| Male | 35 (54.7) |
| Female | 29 (45.3) |
| Disease, n (%) | |
| ALL | 12 (18.8) |
| AML | 28 (43.8) |
| CML | 4 (6.3) |
| CMML | 2 (3.1) |
| Myelofibrosis | 5 (7.8) |
| Lymphoma | 5 (7.8) |
| MDS | 6 (9.4) |
| SAA | 2 (3.1) |
| Type of AHSCT donor, n (%) | |
| Haplo/Cord | 27 (42.2) |
| MRD/MUD | 37 (57.8) |
| Conditioning regimen intensity, n (%) | |
| MAC | 52 (81.3) |
| RIC | 12 (18.8) |
| ATG, n (%) | |
| Yes | 17 (26.6) |
| No | 47 (73.4) |
| Post-transplant Cyclophosphamide n (%) | |
| Yes | 45 (70.3) |
| No | 19 (29.7) |
| HC grade, n (%) | |
| 0 | 0 (0) |
| 1 | 0 (0) |
| 2 | 18 (28.1) |
| 3 | 44 (68.8) |
| 4 | 2 (3.1) |
| Baseline BK viral load (copies/mL) | |
| Median | 3.15 × 10⁸ |
| (min-max) | (1300-10¹⁰) |
| Dose level | |
| 1 × 10⁵ CD3 + CTLs/Kg | 17 (26.6) |
| 2 × 10⁵ CD3 + CTLs/Kg | 47 (73.4) |
| CTL score | |
| Median | 28 |
| (min-max) | (0-36) |
| >15 | 40 (62.5) |

Abbreviations: ATG, anti-thymocyte globulin; haplo: haploidentical related donor; MRD, matched-related donor; MUD, matched unrelated donor; MAC, myeloablativeconditioning; RIC, reduced intensity conditioning; ALL, acute lymphoblastic leukemia; AML, acute myeloid leukemia; CML chronic myeloid leukemia; CMML, chronic myelomonocytic leukemia; MDS, myelodysplastic syndromes; SAA, severe aplastic anemia.

The day 14 overall response (CR+PR) was 72%. Seventeen patients achieved CR (26.6%), 29 patients achieved PR (45.3%), 17 patients failed to respond (26.6%) and 1 patient was not evaluable for response (1.6%). Univariate and multivariate analysis was performed for day +14 response including the variables shown in Table 1. Dose level 2×10⁵ CD3+ CTLs/Kg (p=0.052), age below 47 years (p=0.051) and CTL score greater than 15 (p=0.039) were the only candidate variables found in the univariate analysis. CTL score greater than 15 (HR=4.05, p=0.027) and dose of 2×10⁵ CD3+ CTLs/Kg (HR=3.76, p=0.043) were the only independent predictors for day +14 response in the multivariate analysis, supporting the conclusion that the CTL score calculated with the methodology is the most important prognostic factor for clinical response.

### BKV CTL for Progressive Multifocal Leukoencephalopathy (PML)

Between April 2016 and November 2017, 24 consecutive patients with PML received off-the-shelf BKV-CTLs (Table 2 shows the patients' characteristics). The treatment of the first 3 patients in this study was reported in Muftuoglu, NEJM 379:15; 2018. The same methodology was used to select the optimal cytotoxic T-cell line to be administered to a given patient as described above. Patients received repeated CTLs infusions (1 to 12, median 2.5) at monthly intervals until they achieved CR or their condition deteriorated.

TABLE 2
Baseline PML patients and cell line characteristics and outcomes

| Variable | n (%) | 2-year OS |
|---|---|---|
| Age | | |
| median (range) | 60.5 (32-83) | p = 0.79 |
| ≤60 years | 12 (50.0) | 60.6 |
| >60 years | 12 (50.0) | 48.0 |
| Sex | | p = 0.94 |
| female | 6 (25.0) | 55.6 |
| male | 18 (75.0) | 60.5 |
| Recipient of allogeneic SCT | | p = 0.28 |
| no | 18 (75.0) | 44.1 |
| yes | 6 (25.0) | 83.3 |
| Underlaying disease | | p = 0.50 |
| Myeloid malignance | 6 (25.0) | 30.0 |
| Lymphoid malignance | 9 (37.5) | 64.3 |
| HIV | 6 (25.0) | 66.7 |
| Other | 3 (12.5) | 66.7 |
| Modified Rankin score | | p = 0.46 |
| 0-1 | 0 (0.0) | — |
| 2-3 | 5 (21.7) | 80.0 |
| 4-5 | 18 (78.3) | 47.5 |
| CD4 count in peripheral blood | | |
| median (range) cells/μL | 193.5 (22-863) | p = 0.16 |
| ≤200 cells/μL | 9 (50.0) | 71.1 |
| >200 cells/μL | 9 (50.0) | 43.2 |
| CSF viral load | | |
| median (range) | 484 (<72-2.3 × 10⁵) | p = 0.48 |
| <250 copies/mL | 8 (34.8) | 66.7 |
| 250-2500 copies/mL | 7 (30.4) | 62.5 |
| >2500 copies/mL | 8 (34.8) | 46.7 |
| HLA match | | p = 0.08 |
| 1 | 5 (20.8) | 0.0 |
| 2 | 11 (45.8) | 75.8 |
| 3-4 | 8 (33.3) | 57.1 |
| Cell line selection score | | p = 0.009 |
| ≤10 | 5 (20.8) | 0.0 |
| >10 | 19 (79.2) | 80.0 |

OS is Overall Survival

During the study, 14 (58.3%) patients achieved PR (median time 1.1 months, 95 CI 0.5-1.7) and 9 (37.5%) patients archived CR (median time 7.0 months, 95 CI 3.7-10.3). The 12-month cumulative incidences of PR and CR were 67.7% (95 CI 41.4-85.2) and 53.0% (95 CI 27.2-73.4), respectively. Clinical response was associated with radiological improvement.

The influence of the variables included in Table-2 on outcome was examined. Interestingly, clinical variables such as the underlying cause of immunosuppression, the baseline CD4 count, the baseline CSF viral load, or the modified Rankin score did not have a significant impact on OS or on the achievement of response. The 19 patients who received cell lines with a patient-cell line scores >10 points had a significantly superior overall survival (79.2%) than the 5 patients who received lines with scores ≤10 points (0.0%, p=0.009), supporting the notion that our methodology is capable to select the CTL more likely to result in clinical responses.

### SARS-CoV-2-Specific T Cells for the Treatment of COVID-19 in Immunocompromised Cancer Patients

Between January 2021 and June 2022, 26 consecutive cancer patients with COVID-19 received off-the-shelf SARS-CoV-2-specific CTLs (Table 3 shows the patients' characteristics). For each patient, the CTLs were chosen from the SARS-CoV-2-specific CTL bank using the method to select the optimal cytotoxic T-cell line to be administered to a given patient by calculating a score that takes into consideration several factors, such as the specific HLA matches between the cell line and the patient, as well as measurements of the potency of the cell line, and then choosing for each patient the cell line with the highest score. Patients received repeated CTLs infusions (1 to 2, median 1) at biweekly intervals until they responded or their condition deteriorated.

TABLE 3
Baseline characteristics of the 26 COVID19 patients

| Variable | n (%) |
|---|---|
| Age | |
| median (range) | 54.5 (23-82) |
| Sex | |
| female | 8 (30.8) |
| male | 18 (69.2) |
| Recipient of allogeneic SCT | |
| no | 12 (46.2) |
| yes | 14 (53.8) |
| Underlaying disease | |
| AML/MDS | 8(30.8) |
| CML/MF | 4 (15.4) |
| ALL | 2 (7.7) |
| NHL | 9 (34.6) |
| Lymphoid other | 2 (7.7) |
| Prostate cancer | 1 (3.8) |
| Clinical characteristics | |
| Requiring Oxygen | 5 (19.2) |
| Multifocal pneumonia | 23 (88.5) |
| Number of CTL infusions | |
| 1 | 24 (92.3) |
| 2 | 2 (7.7) |
| CTLs cell dose | |
| 2 × 10⁵ CD3 + CTLs/Kg | 26 (100) |

Twenty-four out of 25 (96%) evaluable patients achieved a response, defined as improvement by at least one grade on the WHO scale. The median time to response was 17 days. There was no toxicity associated with the administration of the CTLs. As 24 out of 25 patients responded, statistical analysis could not be performed to identify prognostic factors for response, such us CTL score; however, in particular embodiments this data supports that CTLs selected with the methodology are highly efficacious.

### Adenovirus-Specific T Cells for the Treatment of Adenovirus Infection in Immunocompromised Cancer Patients

Between April 2018 and January 2022, 29 consecutive cancer patients with adenovirus infection received off-the-shelf adenovirus-specific CTLs (Table 4 shows the patients' characteristics). For each patient, the CTLs were chosen from an adenovirus-specific CTL bank using the method to select the optimal cytotoxic T-cell line to be administered to a given patient by calculating a score that takes into consideration several factors, such as the specific HLA matches between the cell line and the patient, as well as measurements of the potency of the cell line, and then choosing for each patient the cell line with the highest score. Patients received repeated CTLs infusions (1 to 5, median 1) at biweekly intervals until they responded or their condition deteriorated.

TABLE 4
Baseline characteristics of the 29 adenovirus patients

| Variable | n (%) |
|---|---|
| Age | |
| median (range) | 47 (9-66) |
| Sex | |
| female | 13 (44.8) |
| male | 16 (55.2) |
| Recipient of allogeneic SCT | |
| no | 1 (3.4) |
| yes | 28 (96.6) |
| Underlaying disease | |
| AML/MDS | 11(38.0) |
| CML/MF | 6 (20.7) |
| ALL | 5 (17.2) |
| Lymphoma | 5 (17.2) |
| CLL/MM | 2 (6.9) |
| Site of adenovirus infection | |
| Pneumonia | 2 (6.9) |
| Nephritis | 2 (6.9) |
| Cystitis | 1 (3.4) |
| Viremia | 24 (82.8) |
| Viral load (copys) | |
| median (range) | 3.4e5 (0-4.7e7) |
| Number of CTL infusions | |
| 1 | 22 (75.9) |
| 2 | 3 (10.3) |
| 3 o more | 4 (13.8) |
| CTLs cell dose | |
| 2 × 10⁵ CD3 + CTLs/Kg | 29 (100) |

Twenty three out of 26 (88.5%) evaluable patients achieved a response. There was no toxicity associated with the administration of the CTLs. Again, statistical analysis could not be performed to identify prognostic factors for response, such as CTL score, because of the overwhelming proportion of responders, supporting the indication than CTLs selected with the methodology are highly efficacious.

It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Although the present disclosure and certain representative advantages have been described in detail, it should be understood that various changes, substitutions, and alterations can be made herein without departing from the spirt and scope of the present disclosure. For example, the method may be implemented as a computer readable medium, such as a non-transitory computer readable medium, or in an apparatus, such as an information handling system. Additionally, the disclosed method may be implemented using logical operations. In some embodiments, the logical operations may be embedded on an application specific integrated circuit (ASIC) or a very large scale integrated (VLSI) circuit. In another embodiment, the apparatus may include a processor, memory, and/or another component, such as a personal data assistance (PDAs), multi-processor servers, or another comparable device. In some embodiments, the aspects of the invention may be applied to the design of or implemented on different kinds of processors, such as graphics processing unit (GPUs), central processing units (CPUs), and digital signal processors (DSPs).

One of ordinary skill in the art will recognize, however, that embodiments of the disclosure may be practiced without one or more of the specific details, or with other methods, components, materials, and so forth. In other instances, well-known structures, materials, or operations are not shown or described in detail to avoid obscuring aspects of the disclosure.

All of the methods disclosed and claimed herein can be made and executed without undue experimentation in light of the present disclosure. While the compositions and methods of this disclosure have been described in terms of particular embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method, comprising:
receiving, by a processor, patient information representative of one or more human leukocyte antigen (HLA) genes for a patient; and
determining, by the processor using an algorithm, a viral-specific cytotoxic T-cell line suitable for administering to the patient based on a cell line identification number for each of a plurality of cell lines that may be selected for the patient, and based on information about the HLA gene(s) indicated in the patient information.

2. The method of claim 1, wherein the determining step is further defined as determining a viral-specific cytotoxic T-cell line suitable for administering to the patient based on values representative of matching of HLA gene(s) of the patient with respect to corresponding HLA gene(s) in the plurality of cell lines.

3. The method of claim 1 or 2, wherein the information about the HLA gene(s) for the patient comprises, for each of the two chromosomes, the identity of the HLA gene, the HLA group, and the specific HLA protein.

4. The method of claim 1, 2 or 3, wherein the information of the HLA gene(s) for the patient is represented by the following expression, HLA-X*YY:ZZ
where X is an HLA gene, YY is an HLA group, and ZZ is a specific HLA protein.

5. The method of claim 4, wherein the information of the HLA gene(s) is determined by a binary selection at YY and ZZ, said binary selection representing an allele from each of two chromosomes of the patient.

6. The method of claim 4 or 5, wherein in the formula, X represents HLA-A, HLA-B, HLA-C, or HLA-DR.

7. The method of any one of claims 4-6, wherein the patient information about the HLA gene comprises information with respect to a binary selection at YY and ZZ for each of HLA-A, HLA-B, HLA-C, and HLA-DR.

8. The method of any one of claims 4-7, wherein the determining by the processor of a viral-specific cytotoxic T-cell line suitable for administering to the patient based on the cell line identification number is further defined as identifying a T-cell line having the same HLA allele for one or both chromosomes for one or more of HLA-A, HLA-B, HLA-C, and HLA-DR.

9. The method of any one of claims 1-8, wherein determining by the processor the viral-specific cytotoxic T-cell line from a plurality of cell lines that may be selected for the patient comprises receiving information about a CD4+ or CD8+ dominant response based on information about an HLA gene for the patient.

10. The method of any one of claims 2-9, wherein when the response to the virus corresponding to the viral-specific cytotoxic T cell line is CD4 dominant, matching at HLA-DR has a weighted value for the algorithm that is greater than a weighted value for matching at HLA-B and/or a weighted value for matching at HLA-C.

11. The method of any one of claims 2-10, wherein when the response to the virus corresponding to the viral-specific cytotoxic T cell line is CD4 dominant, matching at HLA-A has a weighted value for the algorithm that is greater than a weighted value for matching at HLA-B and/or a weighted value for matching at HLA-C.

12. The method of any one of claims 2-11, wherein when the response to the virus corresponding to the viral-specific cytotoxic T cell line is CD4 dominant, the combination of matching at one allele for one chromosome at HLA-DR and matching at one allele for one chromosome at HLA-A has a weighted value that is greater than a weighted value for the combination of matching at each allele for both chromosomes of HLA-A or for both chromosomes at HLA-DR.

13. The method of any one of claims 2-12, wherein when the response to the virus corresponding to the viral-specific cytotoxic T cell line is CD4 dominant, matching at HLA-DR in the absence of concomitant matching at HLA-A has a weighted value that is greater than a weighted value for matching at HLA-A with no concomitant matching at HLA-DR.

14. The method of any one of claims 2-13, wherein when the response to the virus corresponding to the viral-specific cytotoxic T cell line is CD8 dominant, matching at HLA-DR has a weighted value for the algorithm that is greater than a weighted value for matching at HLA-B and/or a weighted value for matching at HLA-C.

15. The method of any one of claims 2-14, wherein when the response to the virus corresponding to the viral-specific cytotoxic T cell line is CD8 dominant, matching at HLA-A has a weighted value for the algorithm that is greater than a weighted value for matching at HLA-B and/or a weighted value for matching at HLA-C.

16. The method of any one of claims 2-15, wherein when the response to the virus corresponding to the viral-specific cytotoxic T cell line is CD8 dominant:
the combination of (a) matching at one allele for HLA-DR and (b) matching at one allele for HLA-A has a weighted value in the algorithm that is greater than a weighted value for (1) or (2):
(1) the combination of (a) matching at both alleles of HLA-A and (b) matching at no alleles at HLA-DR, or
(2) the combination of (a) matching at both alleles at HLA-DR and (b) matching at no alleles at HLA-A.

17. The method of any one of claims 2-16, wherein when the response to the virus corresponding to the viral-specific cytotoxic T cell line is CD8 dominant, matching at HLA-DR in the absence of concomitant matching at HLA-A has a weighted value that is less than a weighted value for matching at HLA-A with no concomitant matching at HLA-DR.

18. The method of any one of claims 1-17, further comprising:
receiving, by the processor, information representative of a frequency of reactive cytotoxic T-lymphocyte (CTLs) from a plurality of cell lines that may be selected for the patient, wherein the CTLs comprise CD4+ cells and CD8+ cells and are measured by at least one of interferon-γ (IFN) secreted from CD4+ cells, IFN secreted from CD8+ cells, interleukin (IL)-2 secreted from CD4+ cells, and IL-2 secreted from CD8+ cells,
wherein the step of determining, by the processor, the viral-specific cytotoxic T-cell line from a plurality of cell lines that may be selected for the patient is based on the frequency of reactive CTLs.

19. The method of claim 18, wherein the method further comprises subjecting a plurality of cells comprising CD4+ cells and CD8+ cells, from a plurality of cell lines that may be selected for the patient, to peptides from a viral antigen and identifying a proportion of the cells that are activated by the peptides.

20. The method of claim 19, wherein the determining step is further defined as determining a proportion of CD4+ cells that are activated by the peptides and determining a proportion of CD8+ cells that are activated by the peptides.

21. The method of claim 19 or 20, wherein the activation is determined by measuring the level of secretion of interleukin (IL)-2 and interferon-γ (IFN) from both of the CD4+ and CD8+ cell types.

22. The method of claim 21, wherein the step of determining by the processor the viral-specific cytotoxic T-cell line from a plurality of cell lines that may be selected for the patient comprises identifying among CD4+ cells and CD8+ cells the proportion of CD4+ cells that secrete IL-2 and IFN and the proportion of CD8+ cells that secrete IL-2 and IFN.

23. The method of claim 22, wherein when the virus corresponding to the viral-specific cytotoxic T cell line is CD4 dominant, then there is a greater weighted value for the algorithm for the higher secretion level of CD4+ IFN or CD4+ IL-2.

24. The method of claim 22 or 1823 wherein when the virus corresponding to the viral-specific cytotoxic T cell line is CD8 dominant, then there is a greater weighted value for the algorithm for the higher secretion level of CD8+ IFN or CD8+ IL-2.

25. The method of any one of claims 1-24, wherein determining by the processor a viral-specific cytotoxic T-cell line suitable for administering to the patient based on the cell line identification number is based at least in part on one or more of the following values:
(I) for when the response to the virus is CD4-dominant:
(a) the product of the number of matches at HLA-DR times seven;
(b) the product of the number of matches at HLA-A times six;
(c) the number of matches at HLA-B;
(d) the number of matches at HLA-C;
(e) the number fifteen if there is a match at HLA-DR and HLA-A;
(f) the number one if there is a match at HLA-B and HLA-C;
(g) subtraction of one per integer when the frequency of reactive CD4+ or CD8+ cells is less than 10%;
(h) subtraction of five when the frequency of reactive CD4+ or CD8+ cells is less than 3%;
(i) subtraction of all points if the frequencies of reactive CD4+ or CD8+ cells are <1%; or
(II) for when the response to the virus is CD8-dominant:
(a) product of the number of matches at HLA-A times seven;
(b) product of the number of matches at HLA-DR times 6;
(c) the number of matches at HLA-B;
(d) the number of matches at HLA-C;
(e) the number fifteen if there is a match at HLA-DR and HLA-A;
(f) the number one if there is a match at HLA-B and HLA-C;
(g) subtraction of one per integer when the frequency of reactive CD4+ or CD8+ cells is less than 10%;
(h) subtraction of five when the frequency of reactive CD4+ or CD8+ cells is less than 3%;
(i) subtraction of all points if the frequencies of reactive CD4+ or CD8+ cells are <1%.

26. The method of claim 25, wherein based on the HLA match between the patient and the viral-specific cytotoxic T cell line, (g) and (h) are further defined as subtraction of one per integer:
(a) based on the higher frequency of CD4+ cells that secrete IFN or IL-2; or
(b) based on the higher frequency of CD8+ cells that secrete IFN or IL-2.

27. The method of any one of claims 1-26, wherein a therapeutically effective amount of cells from the cell line having the highest cell line identification number based on the patient information is delivered to the patient.

28. An apparatus, comprising:
a memory; and
a processor coupled to the memory, in which the processor is configured to perform the steps comprising:
receiving, by the processor, patient information representative of one or more human leukocyte antigen (HLA) genes for a patient; and
determining, by the processor using an algorithm, a viral-specific cytotoxic T-cell line suitable for administering to the patient based on a cell line identification number for each of a plurality of cell lines that may be selected for the patient, and based on information about the HLA gene(s) indicated in the patient information.

29. The apparatus of claim 28, wherein the determining step is further defined as determining a viral-specific cytotoxic T-cell line suitable for administering to the patient based on values representative of matching of HLA gene(s) of the patient with respect to corresponding HLA gene(s) in the plurality of cell lines.

30. The apparatus of claim 28 or 29, wherein the information about the HLA gene(s) for the patient comprises, for each of the two chromosomes, the identity of the HLA gene, the HLA group, and the specific HLA protein.

31. The apparatus of claim 28, 29 or 30, wherein the information of the HLA gene(s) for the patient is represented by the following expression, HLA-X*YY:ZZ
where X is an HLA gene, YY is an HLA group, and ZZ is a specific HLA protein.

32. The apparatus of claim 31, wherein the information of the HLA gene(s) is determined by a binary selection at YY and ZZ, said binary selection representing an allele from each of two chromosomes of the patient.

33. The apparatus of claim 31 or 32, wherein in the formula, X represents HLA-A, HLA-B, HLA-C, or HLA-DR.

34. The apparatus of any one of the claims 31-33, wherein the patient information about the HLA gene comprises information with respect to a binary selection at YY and ZZ for each of HLA-A, HLA-B, HLA-C, and HLA-DR.

35. The apparatus of any one of the claims 31-34, wherein the determining by the processor of a viral-specific cytotoxic T-cell line suitable for administering to the patient based on the cell line identification number is further defined as identifying a T-cell line having the same HLA allele for one or both chromosomes for one or more of HLA-A, HLA-B, HLA-C, and HLA-DR.

36. The apparatus of any one of claims 28-35, wherein determining by the processor the viral-specific cytotoxic T-cell line from a plurality of cell lines that may be selected for the patient comprises receiving information about a CD4+ or CD8+ dominant response based on information about an HLA gene for the patient.

37. The apparatus of any one of the claims 29-36, wherein when the response to the virus corresponding to the viral-specific cytotoxic T cell line is CD4 dominant, matching at HLA-DR has a weighted value for the algorithm that is greater than a weighted value for matching at HLA-B and/or a weighted value for matching at HLA-C.

38. The apparatus of any one of the claims 29-37, wherein when the response to the virus corresponding to the viral-specific cytotoxic T cell line is CD4 dominant, matching at HLA-A has a weighted value for the algorithm that is greater than a weighted value for matching at HLA-B and/or a weighted value for matching at HLA-C.

39. The apparatus of any one of the claims 29-38, wherein when the response to the virus corresponding to the viral-specific cytotoxic T cell line is CD4 dominant, the combination of matching at one allele for one chromosome at HLA-DR and matching at one allele for one chromosome at HLA-A has a weighted value that is greater than a weighted value for the combination of matching at each allele for both chromosomes of HLA-A or for both chromosomes at HLA-DR.

40. The apparatus of any one of claims 29-39, wherein when the response to the virus corresponding to the viral-specific cytotoxic T cell line is CD4 dominant, matching at HLA-DR in the absence of concomitant matching at HLA-A has a weighted value that is greater than a weighted value for matching at HLA-A with no concomitant matching at HLA-DR.

41. The apparatus of any one of the claims 29-40, wherein when the response to the virus corresponding to the viral-specific cytotoxic T cell line is CD8 dominant, matching at HLA-DR has a weighted value for the algorithm that is greater than a weighted value for matching at HLA-B and/or a weighted value for matching at HLA-C.

42. The apparatus of any one of the claims 29-41, wherein when the response to the virus corresponding to the viral-specific cytotoxic T cell line is CD8 dominant, matching at HLA-A has a weighted value for the algorithm that is greater than a weighted value for matching at HLA-B and/or a weighted value for matching at HLA-C.

43. The apparatus of any one of the claims 29-42, wherein when the response to the virus corresponding to the viral-specific cytotoxic T cell line is CD8 dominant:
the combination of (a) matching at one allele for HLA-DR and (b) matching at one allele for HLA-A has a weighted value in the algorithm that is greater than a weighted value for (1) or (2):
(1) the combination of (a) matching at both alleles of HLA-A and (b) matching at no alleles at HLA-DR, or
(2) the combination of (a) matching at both alleles at HLA-DR and (b) matching at no alleles at HLA-A.

44. The apparatus of any one of the claims 29-43, wherein when the response to the virus corresponding to the viral-specific cytotoxic T cell line is CD8 dominant, matching at HLA-DR in the absence of concomitant matching at HLA-A has a weighted value that is less than a weighted value for matching at HLA-A with no concomitant matching at HLA-DR.

45. The apparatus of any of the claims 29-44, wherein the processor is further configured to perform the steps comprising:
receiving, by the processor, information representative of a frequency of reactive cytotoxic T-lymphocyte (CTLs) from a plurality of cell lines that may be selected for the patient, wherein the CTLs comprise CD4+ cells and CD8+ cells and are measured by at least one of interferon-γ (IFN) secreted from CD4+ cells, IFN secreted from CD8+ cells, interleukin (IL)-2 secreted from CD4+ cells, and IL-2 secreted from CD8+ cells,
wherein the step of determining, by the processor, the viral-specific cytotoxic T-cell line from a plurality of cell lines that may be selected for the patient is based on the frequency of reactive CTLs.

46. The apparatus of claim 45, wherein the processor is further configured to perform the step comprising subjecting a plurality of cells comprising CD4+ cells and CD8+ cells, from a plurality of cell lines that may be selected for the patient, to peptides from a viral antigen and identifying a proportion of the cells that are activated by the peptides.

47. The apparatus of claim 46, wherein the determining step is further defined as determining a proportion of CD4+ cells that are activated by the peptides and determining a proportion of CD8+ cells that are activated by the peptides.

48. The apparatus of claim 45 or 46, wherein the activation is determined by measuring the level of secretion of interleukin (IL)-2 and interferon-gamma (IFN) from both of the CD4+ and CD8+ cell types.

49. The apparatus of claim 48, wherein the step of determining by the processor the viral-specific cytotoxic T-cell line from a plurality of cell lines that may be selected for the patient comprises identifying among CD4+ cells and CD8+ cells the proportion of CD4+ cells that secrete IL-2 and IFN and the proportion of CD8+ cells that secrete IL-2 and IFN.

50. The apparatus of claim 49, wherein when the virus corresponding to the viral-specific cytotoxic T cell line is CD4 dominant, then there is a greater weighted value for the algorithm for the higher secretion level of CD4+ IFN or CD4+ IL-2.

51. The apparatus of any one of claims 28-50, wherein determining by the processor a viral-specific cytotoxic T-cell line suitable for administering to the patient based on the cell line identification number is based at least in part on one or more of the following values:
(I) for when the response to the virus is CD4-dominant:
(a) the product of the number of matches at HLA-DR times seven;
(b) the product of the number of matches at HLA-A times six;
(c) the number of matches at HLA-B;
(d) the number of matches at HLA-C;
(e) the number fifteen if there is a match at HLA-DR and HLA-A;
(f) the number one if there is a match at HLA-B and HLA-C;
(g) subtraction of one per integer when the frequency of reactive CD4+ or CD8+ cells is less than 10%;
(h) subtraction of five when the frequency of reactive CD4+ or CD8+ cells is less than 3%;
(i) subtraction of all points if the frequencies of reactive CD4+ or CD8+ cells are <1%; or
(II) for when the response to the virus is CD8-dominant:
(a) product of the number of matches at HLA-A times seven;
(b) product of the number of matches at HLA-DR times 6;
(c) the number of matches at HLA-B;
(d) the number of matches at HLA-C;
(e) the number fifteen if there is a match at HLA-DR and HLA-A;
(f) the number one if there is a match at HLA-B and HLA-C;
(g) subtraction of one per integer when the frequency of reactive CD4+ or CD8+ cells is less than 10%;
(h) subtraction of five when the frequency of reactive CD4+ or CD8+ cells is less than 3%;
(i) subtraction of all points if the frequencies of reactive CD4+ or CD8+ cells are <1%.

52. The apparatus of claim 51, wherein based on the HLA match between the patient and the viral-specific cytotoxic T cell line, (g) and (h) are further defined as subtraction of one per integer:
(a) based on the higher frequency of CD4+ cells that secrete IFN or IL-2; or (b) based on the higher frequency of CD8+ cells that secrete IFN or IL-2.

53. The apparatus of any one of claims 28-52, wherein a therapeutically effective amount of cells from the cell line having the highest cell line identification number based on the patient information is delivered to the patient.

54. A computer program product comprising:
a non-transitory computer readable medium comprising instructions for causing an information handling system to perform the steps comprising:
receiving patient information representative of one or more human leukocyte antigen (HLA) genes for a patient; and
determining using an algorithm a viral-specific cytotoxic T-cell line suitable for administering to the patient based on a cell line identification number for each of a plurality of cell lines that may be selected for the patient, and based on information about the HLA gene(s) indicated in the patient information.

55. The computer program product of claim 54, wherein the determining step is further defined as determining a viral-specific cytotoxic T-cell line suitable for administering to the patient based on values representative of matching of HLA gene(s) of the patient with respect to corresponding HLA gene(s) in the plurality of cell lines.

56. The computer program product of claim 54 or 55, wherein the information about the HLA gene(s) for the patient comprises, for each of the two chromosomes, the identity of the HLA gene, the HLA group, and the specific HLA protein.

57. The computer program product of claim 54, 55 or 56, wherein the information of the HLA gene(s) for the patient is represented by the following expression, HLA-X*YY:ZZ
where X is an HLA gene, YY is an HLA group, and ZZ is a specific HLA protein.

58. The computer program product of claim 57, wherein the information of the HLA gene(s) is determined by a binary selection at YY and ZZ, said binary selection representing an allele from each of two chromosomes of the patient.

59. The computer program product of claim 57 or 58, wherein in the formula, X represents HLA-A, HLA-B, HLA-C, or HLA-DR.

60. The computer program product of any one of the claims 57-59, wherein the patient information about the HLA gene comprises information with respect to a binary selection at YY and ZZ for each of HLA-A, HLA-B, HLA-C, and HLA-DR.

61. The computer program product of any one of the claims 57-60, wherein the determining of a viral-specific cytotoxic T-cell line suitable for administering to the patient based on the cell line identification number is further defined as identifying a T-cell line having the same HLA allele for one or both chromosomes for one or more of HLA-A, HLA-B, HLA-C, and HLA-DR.

62. The computer program product of any one of claims 54-61, wherein determining the viral-specific cytotoxic T-cell line from a plurality of cell lines that may be selected for the patient comprises receiving information about a CD4+ or CD8+ dominant response based on information about an HLA gene for the patient.

63. The computer program product of any one of the claims 55-62, wherein when the response to the virus corresponding to the viral-specific cytotoxic T cell line is CD4 dominant, matching at HLA-DR has a weighted value for the algorithm that is greater than a weighted value for matching at HLA-B and/or a weighted value for matching at HLA-C.

64. The computer program product of any one of the claims 55-63, wherein when the response to the virus corresponding to the viral-specific cytotoxic T cell line is CD4 dominant, matching at HLA-A has a weighted value for the algorithm that is greater than a weighted value for matching at HLA-B and/or a weighted value for matching at HLA-C.

65. The computer program product of any one of the claims 55-64, wherein when the response to the virus corresponding to the viral-specific cytotoxic T cell line is CD4 dominant, the combination of matching at one allele for one chromosome at HLA-DR and matching at one allele for one chromosome at HLA-A has a weighted value that is greater than a weighted value for the combination of matching at each allele for both chromosomes of HLA-A or for both chromosomes at HLA-DR.

66. The computer program product of any one of claims 55-65, wherein when the response to the virus corresponding to the viral-specific cytotoxic T cell line is CD4 dominant, matching at HLA-DR in the absence of concomitant matching at HLA-A has a weighted value that is greater than a weighted value for matching at HLA-A with no concomitant matching at HLA-DR.

67. The computer program product of any one of the claims 55-66, wherein when the response to the virus corresponding to the viral-specific cytotoxic T cell line is CD8 dominant, matching at HLA-DR has a weighted value for the algorithm that is greater than a weighted value for matching at HLA-B and/or a weighted value for matching at HLA-C.

68. The computer program product of any one of the claims 55-67, wherein when the response to the virus corresponding to the viral-specific cytotoxic T cell line is CD8 dominant, matching at HLA-A has a weighted value for the algorithm that is greater than a weighted value for matching at HLA-B and/or a weighted value for matching at HLA-C.

69. The computer program product of any one of the claims 55-68, wherein when the response to the virus corresponding to the viral-specific cytotoxic T cell line is CD8 dominant:
the combination of (a) matching at one allele for HLA-DR and (b) matching at one allele for HLA-A has a weighted value in the algorithm that is greater than a weighted value for (1) or (2):
(1) the combination of (a) matching at both alleles of HLA-A and (b) matching at no alleles at HLA-DR, or
(2) the combination of (a) matching at both alleles at HLA-DR and (b) matching at no alleles at HLA-A.

70. The computer program product of any one of the claims 55-69, wherein when the virus corresponding to the viral-specific cytotoxic T cell line is CD8 dominant, matching at HLA-DR in the absence of concomitant matching at HLA-A has a weighted value that is less than a weighted value for matching at HLA-A with no concomitant matching at HLA-DR.

71. The computer program product of any of the claims 55-70, wherein the non-transitory computer readable medium is further configured to perform the instructions comprising:
receiving information representative of a frequency of reactive cytotoxic T-lymphocyte (CTLs) from a plurality of cell lines that may be selected for the patient, wherein the CTLs comprise CD4+ cells and CD8+ cells and are measured by at least one of interferon-γ (IFN) secreted from CD4+ cells, IFN secreted from CD8+ cells, interleukin (IL)-2 secreted from CD4+ cells, and IL-2 secreted from CD8+ cells,
wherein the step of determining the viral-specific cytotoxic T-cell line from a plurality of cell lines that may be selected for the patient is based on the frequency of reactive CTLs.

72. The apparatus of claim 71, wherein the non-transitory computer readable medium is further configured to perform the step comprising subjecting a plurality of cells comprising CD4+ cells and CD8+ cells, from a plurality of cell lines that may be selected for the patient, to peptides from a viral antigen and identifying a proportion of the cells that are activated by the peptides.

73. The apparatus of claim 72, wherein the determining step is further defined as determining a proportion of CD4+ cells that are activated by the peptides and determining a proportion of CD8+ cells that are activated by the peptides.

74. The apparatus of claim 72 or 73, wherein the activation is determined by measuring the level of secretion of interleukin (IL)-2 and interferon-γ (IFN) from both of the CD4+ and CD8+ cell types.

75. The apparatus of claim 74, wherein the step of determining the viral-specific cytotoxic T-cell line from a plurality of cell lines that may be selected for the patient comprises identifying among CD4+ cells and CD8+ cells the proportion of CD4+ cells that secrete IL-2 and IFN and the proportion of CD8+ cells that secrete IL-2 and IFN.

76. The apparatus of claim 75, wherein when the response to the virus corresponding to the viral-specific cytotoxic T cell line is CD4 dominant, then there is a greater weighted value for the algorithm for the higher secretion level of CD4+ IFN or CD4+ IL-2.

77. The apparatus of any one of claims 54-76, wherein determining a viral-specific cytotoxic T-cell line suitable for administering to the patient based on the cell line identification number is based at least in part on one or more of the following values:
(I) for when the response to the virus is CD4-dominant:
(a) the product of the number of matches at HLA-DR times seven;
(b) the product of the number of matches at HLA-A times six;
(c) the number of matches at HLA-B;
(d) the number of matches at HLA-C;
(e) the number fifteen if there is a match at HLA-DR and HLA-A;
(f) the number one if there is a match at HLA-B and HLA-C;
(g) subtraction of one per integer when the frequency of reactive CD4+ or CD8+ cells is less than 10%;
(h) subtraction of five when the frequency of reactive CD4+ or CD8+ cells is less than 3%; and
(i) subtraction of all points if the frequencies of reactive CD4+ or CD8+ cells are <1%; or
(II) for when the response to the virus is CD8-dominant:
(a) product of the number of matches at HLA-A times seven;
(b) product of the number of matches at HLA-DR times 6;
(c) the number of matches at HLA-B;
(d) the number of matches at HLA-C;
(e) the number fifteen if there is a match at HLA-DR and HLA-A;
(f) the number one if there is a match at HLA-B and HLA-C;
(g) subtraction of one per integer when the frequency of reactive CD4+ or CD8+ cells is less than 10%;
(h) subtraction of five when the frequency of reactive CD4+ or CD8+ cells is less than 3%;
(i) subtraction of all points if the frequencies of reactive CD4+ or CD8+ cells are <1%.

78. The apparatus of claim 77, wherein based on the HLA match between the patient and the viral-specific cytotoxic T cell line, (g) and (h) are further defined as subtraction of one per integer:
(a) based on the higher frequency of CD4+ cells that secrete IFN or IL-2; or
(b) based on the higher frequency of CD8+ cells that secrete IFN or IL-2.

79. The apparatus of any one of claims 54-78, wherein a therapeutically effective amount of cells from the cell line having the highest cell line identification number based on the patient information is delivered to the patient.
